(19) United States
(12) Patent Application Publication
(10) Pub. No.: US 2009/0221430 A1
(43) Pub. Date: Sep. 3, 2009

(54) Proteomic Methods For The Identification And Use Of Putative Biomarkers Associated With The Dysplastic State In Cervical Cells Or Other Cell Types

(75) Inventors: Shiaw-Lin Wu, Weston, MA (US); William S. Hancock, Brookline, MA (US); Barry L. Karger, Newton, MA (US); James Linder, Ohama, NE (US); David W. Hanlon, Derry, NH (US)

Correspondence Address: WEINGARTEN, SCHURGIN, GAGNEBIN & LEBOVICI LLP, TEN POST OFFICE SQUARE, BOSTON, MA, 02109, US

(73) Assignees: CYTYC CORPORATION, Marlborough, MA (US); NORTHEASTERN UNIVERSITY, Boston, MA (US)

(21) Appl. No.: 12/224,973

(22) PCT Filed: Mar. 12, 2007
(86) PCT No.: PCT/US2007/006176

§ 371 Date: Feb. 23, 2009

Related U.S. Application Data
(60) Provisional application No. 60/780,983, filed on Mar. 10, 2006.

(51) Int. Cl. C40B 30/00; C12Q 1/68; G01N 33/68; G01N 33/536; C12Q 1/02

(52) U.S. Cl. 506/7; 435/6; 436/86; 436/536; 435/29

(57) ABSTRACT

The invention relates to methods for detecting and identifying potential biomarkers of high-grade cervical dysplasia in an individual human subject. The invention also relates to newly discovered biomarkers, as set forth in Tables 1-4 herein, which are associated with the dysplastic state of cervical cells. It has been discovered that a differential level of expression of any of these markers or combination of these markers correlates with a dysplastic condition in a human subject, e.g., a patient.

### CROSS REFERENCE TO RELATED APPLICATIONS

This application claims the priority of U.S. Provisional Application No. 60/780,983, filed Mar. 10, 2006, entitled, PROTEOMIC METHODS FOR THE IDENTIFICATION AND USE OF PUTATIVE BIOMARKERS ASSOCIATED WITH THE DYSPLASTIC STATE IN CERVICAL CELLS OR OTHER CELL TYPES, the whole of which is hereby incorporated by reference herein.

### STATEMENT REGARDING FEDERALLY SPONSORED RESEARCH OR DEVELOPMENT

N/A

### BACKGROUND OF THE INVENTION

Carcinoma of the uterine cervix is the second most common neoplasm among women worldwide, and fifth leading cause of all cancer related deaths (Baldwin et al., 2003). Recent estimates indicate that approximately 500,000 new cases of cervical cancer are diagnosed annually (Munoz et al., 1989; NIH Statement, 1996). Cervical carcinoma develops slowly over a time period of several years through well-defined non-invasive stages. Preneoplastic lesions, classified as cervical intraepithelial neoplasia (CIN), are defined according to the degree of cellular abnormality and have the potential to progress to carcinoma in situ or invasive carcinoma. While only a small fraction of all dysplastic lesions would progress to invasive cervical cancer if left untreated, the overall risk escalates with increased grade of lesion (Melnikow et al., 1998).

It has been well established that early detection of these morphological changes significantly increases the chances for successful treatment. Since its introduction in the 1940's, the conventional Pap test has dramatically decreased the incidence and mortality rates associated with cervical cancer by identifying and classifying cellular changes associated with the progression to cancer. The abnormal morphological changes that precede squamous cell carcinoma have been classified according to numerous systems, including the commonly used 2001 Bethesda System (Tabbara et al., 1992), among others. Under the Bethesda System, the abnormal morphological changes include Atypical Squamous Cells of Undetermined Significance (ASC-US), Atypical Squamous Cells—Cannot Exclude High-Grade (ASC-H), Low-Grade Squamous Intraepithelial Lesions (LSIL) and High-Grade Squamous Intraepithelial Lesions (HSIL). The concept of cervical intraepithelial neoplasia, or CIN, was introduced in 1967 to embrace all grades of dysplasia and carcinoma in situ under a single disease heading. The diagnosis of CIN is based on histological sections and graded as I, II and III. These and other generally accepted classification schemes are more fully described in Chan et al., The Papanicolaou Test—Its Current Status (1990) Hong Kong Practitioner, 12, 1198-1203, which is incorporated by reference herein. More recent liquid-based cytology (LBC) preparations, such as the ThinPrep® Pap Test (Cytyc Corporation), have proven useful in reducing the number of inadequate Pap tests and the incidence of false negative diagnoses by enabling the improved homogeneous transfer of cells from the cervix to the slide (Roberts et al., 1997). The use of computer imaging to locate potential abnormal cells has also improved the detection of preneoplastic lesions of the cervix. Since the introduction of LBC methodology, the American Cancer Society estimates that the rate of invasive cervical cancer in the US has declined by 28%. The success of the Pap test, however, ultimately relies upon the ability of the technician to accurately identify and evaluate those characteristic cellular changes.

Therefore, despite recent improvements in sample collection, processing and image-directed slide review, a number of studies have reported low substantial inter-observer variability and Pap test discordance with histological follow-up, particularly within the ASCUS and LSIL diagnostic categories (Howell et al., 2004; Joste et al., 2005). Importantly, low sensitivity and poor reproducibility within these cytological categories have complicated the management of this subset of patients. Because a diagnosis that is made based upon the cytology sample establishes the basis for further treatment, an inaccurate diagnosis may lead to over-treatment of a healthy woman (i.e., colposcopy and biopsy) or under-treatment of a woman having a cervical lesion. False negative and positive results are therefore costly in terms of time and expense, and can generate significant anxiety in affected women. Improvements in diagnostic accuracy would therefore benefit patients and reduce related health system costs.

The subjectivity of cervical cytology may be reduced by integrating the use of objective markers to help determine the presence and severity of dysplastic cells. For example, high-risk human papillomavirus (HPV) has been shown to be present in 99% of all cervical cancers (Syrjanen et al., 1987), and the concept that persistent viral infection is required for progression to cervical neoplasia is well accepted (Cuschieri et al., 2005). However, while HPV DNA testing can provide an objective measurement, high-risk HPV testing cannot accurately discriminate between patients whose squamous intraepithelial lesions will persist or progress to invasive carcinoma and those whose lesions will regress spontaneously. It was reported in the ASCUS-LSIL Triage Study that 83% of women having an LSIL Pap result tested positive for high-risk HPV (ASCUS-LSIL Triage Study (ALTS) Group, 2003), a level too high to provide clinical utility in a patient triage strategy. Although a triage strategy that incorporates HPV detection within the ASCUS population has proven to be more sensitive for detecting underlying high-grade disease, decreased specificity was a primary concern (Shiffman et al., 2003). HPV screening is currently most appropriate in the triage of borderline or ASCUS cytology cases and in conjunction with Pap testing for women 30 years or older. In other words, in this age group, benefits of HPV testing independent of cytology classification have been observed.

The human papillomavirus contributes to neoplastic progression predominantly through the action of two viral oncoproteins, E6 and E7, which interact with various host regulatory proteins to influence the function or expression levels of host gene products, eventually leading to the disruption of the cell cycle (Shai et al., 2007). It has been previously demonstrated that the E6 oncoprotein interacts with the p53 tumor suppressor protein (Crook et al., 1991), while E7 binds to the retinoblastoma protein, pRb. p16^{INK4a} is a cyclin-dependent kinase inhibitor that negatively regulates cell proliferation by inhibiting hyperphosphorylation of pRb via the cdk4/6 complex. Overexpression of the p16^{INK4a} protein has been well documented in cervical cancer and is a consequence of pRb targeted inactivation from E7. While it has been proposed that p16^{INK4a} is a useful biomarker for the identification of dysplastic cervical epithelial cells, its specificity has been questioned and other surrogate markers may exist that also have clinical utility due to their ability to quantify cellular changes that are indicative of active HPV oncogene expression rather than viral presence only. The differential expression of specific cellular proteins might therefore prove useful in identifying those clinically important cases of HPV infection that have a more significant risk of progression towards cervical carcinoma.

### BRIEF SUMMARY OF THE INVENTION

While molecular tests for the detection of HPV are very sensitive, the specificity of HPV testing is not currently high enough to perform well in a primary screening setting and is therefore most useful in the triage of ASCUS cytology cases. Incorporation of cellular biomarkers indicative of cervical cancer progression to and through the dysplastic state may help improve sensitivity, specificity, standardization and ultimately the quality of diagnosis. More recently, a variety of molecular approaches have been utilized to identify potential markers of cervical cancer. However, in all cases, cultured mammalian cell lines or cervical cancer tissue was utilized for discovery research purposes. Furthermore, the majority of these research efforts evaluated changes in gene expression, which may or may not directly translate to the protein level. Thus, significant opportunities exist for the identification of cervical markers specifically for the dysplastic state and their utilization in the development of convenient to use, robust and predictive tests having improved diagnostic value.

Laser Capture Microdissection (LCM) is a powerful tool that enables the isolation of specific cell types from a heterogeneous population. While this technology has routinely been used with tissue, few studies have applied this methodology to investigate cytological specimens in conjunction with protein analysis. LCM was utilized to select approximately 10,000 high-grade (HSIL) dysplastic cells per specimen from ThinPrep Pap Test prepared slides. Following cell capture, samples were processed and analyzed using a highly sensitive linear ion trap with Fourier transform mass spectrometer (LTQ-FTMS). Multiple individual specimens having a clinical diagnosis of either Within Normal Limits (WNL) or HSIL were evaluated and compared in order to identify proteins that exhibited differential changes in expression, either upregulated and down-regulated.

Described herein are the specimen processing and proteomic methods of the invention, which are used to detect and identify potential biomarkers for cervical dysplasia, and the potential biomarkers for cervical dysplasia identified thereby. These same specimen processing and proteomic analysis methods can also be used to enrich any type of clinical sample, preferably an easily accessible clinical sample, for putative dysplastic cells and to analyze the enriched population for novel biomarkers. Information obtained from this type of analysis would be most useful in identifying protein expression profiles or protein signatures that become apparent in dysplastic conditions, before the cells are committed to the cancerous state. A significant aspect of this invention therefore relates to the proteomic characterization of high-grade dysplastic cells. The differential expression of proteins in high-grade dysplastic cells versus morphologically normal cells (of cervical or other tissue) can lead to the potential identification of novel biomarkers most useful in the detection, diagnosis and stratification of the dysplastic condition.

Thus, in general, the invention provides a method for the identification of biomarkers for the classification of cells in a manner that can complement or replace any cytological or histological analysis. An exemplary method of identifying a potential cervical dysplasia biomarker for the classification of cells in conjunction with a cytological or histological analysis includes the steps of: a) providing a cervical sample from a patient; b) carrying out the cytological or histological analysis on a specimen from the cervical sample; c) marking high-grade dysplastic cells generically identified by the cytological or histological analysis (e.g., Pap Test stained cells); d) carrying out laser capture microdissection (LCM) of the marked cells; e) lysing the captured cells; f) separating the proteins in the lysed cell preparation (e.g., by SDS-PAGE) and digesting the separated proteins (e.g., with trypsin); g) analyzing the digested samples (e.g., by LTQ/FT LC/MS/MS); h) determining a profile of protein abundance in each of the digested samples of marked cells; i) comparing the protein abundance profiles of said high risk patients with similarly determined protein abundance profiles of healthy individuals; and j) identifying any proteins that are present in the abundance profiles of said high risk patients but not in, or at a reduced level in, the abundance profiles of said healthy individuals, wherein any protein so identified is said potential cervical dysplasia biomarker. In a preferred embodiment, the patient from whom the cervical sample is obtained is suspected of being at high risk of developing a cervical cancer.

Furthermore, provided herein, in Tables 1-4, are panels of proteins identified in samples from individual women at risk of developing cervical cancer, wherein the samples have previously been enriched for cells in a dysplastic state. The proteins in these panels, either individually or as relative ratios, are potential biomarkers for the identification of a dysplasia in cervical tissue. Preferentially, the relative ratios of a combination or combinations of biomarkers are utilized for improved diagnostic performance. The methods of the invention also would be useful to detect and to identify potential biomarkers for any dysplastic condition in similarly enriched cell samples.

Using the methods of the invention, potential biomarker proteins for a predisposition to high-grade cervical dysplasia have been characterized in individual subjects. Use of proteins identified according to the principles of the invention as biomarkers for the classification of cervical dysplasia is within the invention. In addition, the invention provides a sensitive method for early detection of dysplasia and for monitoring of the related potentially cancerous state.

Thus, in one aspect, the invention is directed to a method for assessing the presence of a cervical dysplastic lesion in a human subject, the method including comparing the level of abundance, in a sample from the subject, of at least one marker of the invention selected from the group consisting of the markers listed in Tables 1-4; and the normal level of abundance of the at least one marker in a control sample, wherein a significantly higher level of abundance of the at least one marker in the sample from the subject compared to the level of abundance of the at least one marker in the control sample is an indication of the presence of a cervical dysplastic lesion in the subject.

Preferably, the level of abundance of the at least one marker in the sample from the subject is three or more times the abundance level of the at least one marker in the control sample. The level of abundance of the at least one marker can be determined by detecting the amount of marker protein present in the sample, for example by using an assay selected from the group consisting of an antibody based assay, a protein array assay and a mass spectrometry based assay. Alternatively, the level of abundance of the at least one marker can be determined by detecting the amount of mRNA that encodes a marker protein present in the sample. The control sample level of abundance of the at least one marker can be determined from a standard table or curve. In particularly preferred embodiments, a plurality of markers (e.g., three or more or five or more) is detected.

The invention additionally provides a test method for assessing the cervical carcinogenic potential of a compound. This method comprises the steps of: obtaining a sample comprising dysplastic cervical cells; maintaining separate aliquots of the dysplastic cells in the presence and absence of a compound to be tested; and comparing the expressed abundance of a marker of the invention in each of the aliquots. A significantly higher level of expression or abundance of a marker according to the invention in the aliquot maintained in the presence of the compound, relative to that of the aliquot maintained in the absence of the compound, is an indication that the compound possesses cervical carcinogenic potential.

In addition, the invention further provides methods for assessing the potential of a test composition as an inhibitor of the dysplastic state, e.g., in cervical cells, in a patient. These methods comprise the steps of: obtaining a sample comprising dysplastic cervical cells; separately maintaining aliquots of the sample in the presence and absence of a test composition; comparing the abundance of a marker of the invention in each of the aliquots; and identifying a composition as an inhibitor of the dysplastic, e.g., cervical dysplastic, state where the composition significantly lowers the level of expression of a marker of the invention in the aliquot containing the composition relative to the levels of expression of the marker in the presence of the other compositions. Compositions so identified can be administered appropriately to a patient having dysplasia for treating or for inhibiting the further development of the dysplasia.

Markers according to the invention may likewise be used to assess the efficacy of a therapy for inhibiting cervical dysplasia in a patient. In this method, the level of expression of one or more markers of the invention in a pair of samples (one subjected to the therapy, the other not subjected to the therapy) is assessed. As with the method of assessing the potential of test compounds, if the therapy induces a significantly lower level of expression of a marker of the invention, then the therapy can be considered potentially efficacious for inhibiting cervical dysplasia. As above, if samples from a selected patient are used in this method, then alternative therapies can be assessed in vitro in order to select a therapy most likely to be efficacious for inhibiting cervical dysplasia in the patient. Furthermore, the methods of the invention may be used to evaluate a patient before, during and after therapy, for example, to evaluate the reduction in tumor burden.

In another aspect, the invention relates to various diagnostic and test kits for detecting the presence of a marker protein in a subject sample (e.g., a cervical sample). In one embodiment, the invention provides a kit for assessing whether a human subject is afflicted with a cervical dysplasia. The kit comprises one or more reagents for assessing expression of at least one marker of the invention. For antibody-based kits, for example, a kit comprises, e.g., (1) a first antibody (e.g., attached to a solid support) that binds to a marker protein; and, optionally, (2) a second, different antibody that binds to either the protein or the first antibody and is conjugated to a detectable label. In another embodiment, the invention provides a kit for assessing the suitability of a chemical or biologic agent for inhibiting the progression of cells in the dysplastic state to the cancerous state in a patient. Such a kit comprises reagents for assessing expression of at least one marker of the invention and may also comprise one or more of such agents. In a further embodiment, the invention provides kits for assessing the presence of dysplastic cells. Such kits may comprise an antibody, an antibody derivative, or an antibody fragment that binds specifically with a marker protein, or a fragment of the protein. Such kits may also comprise a plurality of antibodies, antibody derivatives, or antibody fragments wherein the plurality of such antibody agents binds specifically with a marker protein, or a fragment of the protein.

Other features and advantages of the invention will be apparent from the following description of the preferred embodiments thereof and from the claims.

### DETAILED DESCRIPTION OF THE INVENTION

The invention relates to methods for detecting and identifying potential biomarkers of high-grade cervical dysplasia in an individual human subject. The invention also relates to newly discovered biomarkers, as set forth in Tables 1-4, which are associated with the dysplastic state of cervical cells. It has been discovered that a differential level of expression of any of these markers or combination of these markers correlates with a dysplastic condition in a human subject, e.g., a patient.

As used herein, each of the following terms has the meaning associated with it in this section.

A “marker” is a protein, or associated gene or other nucleic acid, whose altered level of expression (or abundance) in a tissue or cell from its expression level in normal or healthy tissue or cell is associated with a disease state, such as cancer.

“Proteins of the invention” encompass marker proteins and their fragments; variant marker proteins and their fragments (including those with side chain modifications); peptides and polypeptides comprising an at least 15 amino acid segment of a marker or variant marker protein; and fusion proteins comprising a marker or variant marker protein, or an at least 15 amino acid segment of a marker or variant marker protein.

The term “probe” refers to any molecule that is capable of selectively binding to a specifically intended target molecule, for example, a nucleotide transcript or marker protein. Probes can be either synthesized by one skilled in the art or derived from appropriate biological preparations. For purposes of detection of the target molecule, probes may be specifically designed to be labeled, as described herein. Examples of molecules that can be utilized as probes include, but are not limited to, RNA, DNA, proteins, antibodies, and organic molecules.

A “cervical sample” or “patient cervical sample” comprises cervical cells and/or cervical-associated body fluid obtained from a human subject, e.g., a patient.

A “cervical-associated” body fluid is a fluid that, when in the body of a subject, contacts or passes through cervical cells or into which cervical cells or proteins shed from cervical cells are capable of passing. The cells may be found in a cervical smear collected, for example, by a cervical brush. Exemplary cervical-associated body fluids include blood fluids, lymph, ascitic fluids, gynecological fluids, cystic fluid, urine, and fluids collected by vaginal rinsing.

The “normal” level of expression (or WNL level) of a marker is the level of expression or abundance of the marker in a cervical sample of a subject not afflicted with a cervical dysplasia.

An “over-expression” or “significantly higher level of expression” of a marker refers to an abundance or expression level in a test sample that is greater than the standard error of the assay employed to assess expression, and is preferably at least three, and more preferably four, five or ten times the expression level of the marker in a control sample (e.g., sample from a healthy subjects not having the marker-associated condition) and preferably, the average expression level of the marker in several control samples.

A “significantly lower level of expression” of a marker refers to an abundance or expression level in a test sample that is at least three, and more preferably four, five or ten times lower than the expression level of the marker in a control sample (e.g., sample from a healthy subjects not having the marker-associated condition) and preferably, the average expression level of the marker in several control samples.

A “kit” is any manufacture (e.g., a package or container) comprising at least one reagent, e.g., a probe, for specifically detecting the abundance or expression of a marker of the invention. The kit may be promoted, distributed, or sold as a unit for performing the methods of the present invention.

Unless otherwise specified herein, the terms “antibody” and “antibodies” broadly encompass naturally-occurring forms of antibodies (e.g., IgG, IgA, IgM, IgE) and recombinant antibodies such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. It will be appreciated that the methods and kits of the present invention may also include known cervical dysplasia markers or other materials known to bind to proteins such as small molecules, substrate mimetics, other non-antibody binding proteins, RNA or DNA aptamers, etc.

The present invention is based, in part, on newly identified biomarkers, which are differentially expressed in dysplastic cervical cells as compared to their expression in normal or healthy cervical cells. The enhanced or reduced expression of one or more of these markers in cervical cells is herein correlated with the dysplastic state of the tissue. The invention provides compositions, kits and methods for assessing the dysplastic state of cells (e.g., cells obtained from a human, cultured human cells, archived or preserved human cells and in vivo cells) as well as for treating patients afflicted with the dysplastic state.

The invention thus includes a method of assessing the dysplastic state of cervical cells in a human subject. This method comprises comparing the level of expression of one or more markers of the invention (listed in Tables 1-4) in a cervical sample from a subject (i.e., cervical cells and/or cervical-associated body fluid) and the normal level of expression of the one or more markers in a control, e.g., a human subject not afflicted with cervical dysplasia. For, example, a significantly higher level of expression, or abundance, of the marker in the patient sample as compared to the normal level of expression is an indication that the subject has a dysplastic condition. It is also within the invention to use a combination of the identified biomarkers and to assess the differential expression of these markers as a change in their relative ratios.

Furthermore, the invention encompasses in general an approach to targeted clinical proteomics wherein a potentially cancerous lesion from a patient is sampled and then the sample is enriched for a specific dysplastic cell type. In this way, one can correlate morphological changes in the tissue with biomarkers and establish the relationship of the biomarker to the stage of disease. For example, one can identify a biomarker in a cell type associated with a specific staging of the disease and then carry out imaging of the cell type with an antibody to that protein. In this manner, the antibody can be used as a backup to the cytology procedures and to reduce error rates. Also such an antibody can be used for imaging studies of the distribution of cancerous or precancerous cells as the disease progresses.

Although an immunocytochemistry based assay is described herein, the methods according to the invention also encompass using any other method known to or later developed by those of ordinary skill in the art within a cellular and/or morphological context. For example, the use of immunohistochemistry, flow cytometry, etc., as well as soluble formats (e.g., ELISA) are encompassed herein.

In addition to cervical disease, the methods of the invention have application for other diseases and carcinomas such as those of the breast, lung, colon, anus, stomach, nasal tissue, mouth, esophagus and skin. The expectation from theory and general practice is that all squamous/adeno (i.e., “skin”) derived cancers have a pre-invasive phase. The detection of this pre-invasive phase is dependent on the accessibility of the organ.

The colon has pre-cancerous polyps, the anus has pre-invasive skin changes, and similar esophageal changes are observed. Anal and colon lesions may be detected by direct vision via endoscope or colonoscopy, and esophageal lesions by endoscopy. Cells from these pre-cancerous lesions can be obtained via biopsy or washings.

The method of the invention can also be practiced employing a device in which a membrane based on the Pap smear is used to collect a layer of cells from the cancer tissue (in cervix, mouth, lung, nose, eye, kidney tubules, colon, etc., and the membrane is then transferred to an automated device, such as an LCM device, where the target cells are collected. The target cells can be identified, e.g., by a flourescently labelled antibody discovered in an earlier phase of the study. The sensitivity and specificity of such an assay can be increased by combining the Pap smear membrane aspect with LCM. In this manner, one can generate a total abnormal cell count as well as a histogram of the distribution of label.

The invention also includes an array comprising a marker of the present invention. The array can be used to assay abundance of, e.g., one or more proteins in the array. In one embodiment, the array can be used to assay protein abundance in an individual sample from a patient to ascertain the specificity of proteins in the array. In this manner, a large number of proteins can be simultaneously assayed for expression or abundance level. This allows a profile to be developed showing a battery of proteins specifically expressed in one or more sample sites.

In addition to such qualitative determination, the invention allows the quantitation of protein expression. Thus, not only sample site specificity, but also the level of abundance of a battery of proteins in individual samples is ascertainable. Thus, proteins can be grouped on the basis of their expression site per se and level of expression at that site.

In another embodiment, the array can be used to monitor the time course of expression of one or more proteins in the array. This can occur in various biological contexts, as disclosed herein, related to the development of cervical cancer.

Markers of the invention are selective for as an indication of the presence of a cervical dysplastic lesion. By “an indication of the presence of a cervical dysplastic lesion” it is intended that the marker of interest is overexpressed in high-grade cervical disease but is not overexpressed in conditions classified as WNL, ASCUS, LSIL, CINI, immature metaplastic cells, and other conditions that are not considered to be clinical disease. Thus, detection of the markers of the invention permits the differentiation of samples indicative of underlying high-grade cervical disease from samples that are indicative of benign proliferation, or mild dysplasia. As used herein, “mild dysplasia” refers to LSIL and CINI where no high-grade lesion is present. The methods of the invention also distinguish cells indicative of high-grade disease from normal cells, immature metaplastic cells, and other cells that are not indicative of clinical disease. In this manner, the methods of the invention permit the accurate identification of high-grade cervical disease, even in cases mistakenly classified as normal, CINI, LSIL, or ASCUS by traditional Pap testing (i.e., “false negatives”). In some embodiments, the methods for diagnosing high-grade cervical disease are performed as a reflexive response to an abnormal or atypical Pap smear. That is, the methods of the invention may be performed in response to a patient having an abnormal or atypical Pap smear result. In other aspects of the invention, the methods are performed as a primary screening test for high-grade cervical disease in the general population of women, just as the conventional Pap test is performed currently.

The markers of the invention include any gene or protein that is selectively over expressed in cervical disease, as defined herein above. Such markers are capable of identifying cells within a cytology cell suspension that are an indication of the presence of a cervical dysplastic lesion. The biomarkers of the invention detect cells of CINII conditions and above, but do not detect CINI where there is no underlying high-grade disease.

As discussed above, a significant percentage of patients presenting with Pap smears classified as WNL, CINI, or ASCUS actually have lesions characteristic of high-grade cervical disease. Thus, the methods of the present invention permit the identification of high-grade cervical disease in all patient populations, including these “false negative” patients, and facilitate the detection of rare abnormal cells in a patient sample. The diagnosis can be made independent of cell morphology and HPV infection status, although the methods of the invention can also be used in conjunction with conventional diagnostic techniques, e.g., Pap test, molecular testing for high-risk types of HPV, etc.

Assessing the presence of a cervical dysplastic lesion is intended to include, for example, diagnosing or detecting the presence of cervical disease, monitoring the progression of the disease, and identifying or detecting cells or samples that are indicative of high-grade cervical disease. The terms diagnosing, detecting, and identifying high-grade cervical disease are used interchangeably herein. By “high-grade cervical disease” is intended those conditions classified by colposcopy as premalignant pathology or moderate to severe dysplasia. Underlying high-grade cervical disease includes histological identification of CINII, CINIII and HSIL.

In particular embodiments, the diagnostic methods of the invention comprise collecting a cervical sample from a patient, contacting the sample with at least one antibody specific for a marker of interest, and detecting antibody binding. Samples that exhibit over expression of a marker of the invention, as determined by detection of antibody binding, are deemed positive for high-grade cervical disease. In particular embodiments, the body sample is a monolayer of cervical cells. In some aspects of the invention, the monolayer of cervical cells is provided on a glass slide.

By “body sample” is intended any sampling of cells, tissues, or bodily fluids in which expression of a biomarker can be detected. Examples of such body samples include but are not limited to blood, lymph, urine, gynecological fluids, biopsies, and smears. Body samples may be obtained from a patient by a variety of techniques including, for example, by scraping or swabbing an area or by using a needle to aspirate bodily fluids. Methods for collecting various body samples are well known in the art. In particular embodiments, the body sample comprises cervical fluid or cervical cells, as cervical tissue samples or as cervical cells in suspension, particularly in a liquid-based preparation. In one embodiment, cervical samples are collected according to liquid-based cytology specimen preparation guidelines such as, for example, the ThinPrep® System (Cytyc Corporation, Marlborough, Mass.). Body samples may be transferred to a glass slide for viewing under magnification. Fixative and staining solutions may be applied to the cells on the glass slide for preserving the specimen and for facilitating examination. In one embodiment the cervical sample will be collected and processed to provide a monolayer sample, as set forth in U.S. Pat. No. 5,143,627, herein incorporated by reference.

Any methods available in the art for identification or detection of the markers are encompassed herein. The over expression of a biomarker of the invention can be detected on a nucleic acid level or a protein level. In order to determine over expression, the body sample to be examined may be compared with a corresponding body sample that originates from a healthy person. That is, the “normal” level of expression is the level of expression of the biomarker in cervical cells of a human subject or patient not afflicted with high-grade cervical disease. Such a sample can be present in standardized form. In some embodiments, particularly when the body sample comprises a monolayer of cervical cells, determination of biomarker over expression requires no comparison between the body sample and a corresponding body sample that originates from a healthy person. In this situation, the monolayer of cervical cells from a single patient may contain as few as 1-2 abnormal cells per 50,000 normal cells present. Detection of these abnormal cells, identified by their over expression of a biomarker of the invention, precludes the need for comparison to a corresponding body sample that originates from a healthy person.

Methods for detecting markers of the invention comprise any methods that determine the quantity or the presence of the biomarkers either at the nucleic acid or protein level. Such methods are well known in the art and include but are not limited to western blots, northern blots, southern blots, ELISA, immunoprecipitation, immunofluorescence, flow cytometry, immunocytochemistry, nucleic acid hybridization techniques, nucleic acid reverse transcription methods, and nucleic acid amplification methods. In particular embodiments, over expression of a biomarker is detected on a protein level using, for example, antibodies that are directed against specific biomarker proteins. These antibodies can be used in various methods such as Western blot, ELISA, immunoprecipitation, or immunocytochemistry techniques. Likewise, immunostaining of cervical smears can be combined with conventional Pap stain methods so that morphological information and immunocytochemical information can be obtained. In this manner, the detection of the biomarkers can reduce the high false-negative rate of the Pap smear test and may facilitate mass automated screening.

In another aspect, the invention relates to various diagnostic and test kits. In one embodiment, the invention provides a kit for assessing whether a patient is afflicted with high grade cervical dysplasia. The kit comprises a reagent for assessing expression of a marker of the invention. In another embodiment, the invention provides a kit for assessing the suitability of a chemical or biologic agent for inhibiting cervical dysplasia in a patient. Such kits comprise a reagent for assessing expression of a marker of the invention, and may also comprise one or more of such agents. In a further embodiment, the invention provides kits for assessing the presence of cervical dysplastic cells or treating cervical dysplasia. Such kits comprise an antibody, an antibody derivative, or an antibody fragment that binds specifically with a marker protein, or a fragment of the protein. Such kits may also comprise a plurality of antibodies, antibody derivatives, or antibody fragments wherein the plurality of such antibody agents binds specifically with a marker protein, or a fragment of the protein.

In an alternative embodiment, the invention provides a kit for assessing the presence of high-grade cervical dysplastic cells wherein the kit comprises a nucleic acid probe that binds specifically with a marker nucleic acid or a fragment of the nucleic acid. The kit may also comprise a plurality of probes, wherein each of the probes binds specifically with a marker nucleic acid, or a fragment of the nucleic acid. Suitable reagents for binding with a marker nucleic acid (e.g., a genomic DNA, an mRNA, a spliced mRNA, a cDNA, or the like) include complementary nucleic acids. For example, the nucleic acid reagents may include oligonucleotides (labeled or non-labeled) fixed to a substrate, labeled oligonucleotides not bound with a substrate, pairs of PCR primers, molecular beacon probes, and the like.

For antibody-based kits, the kit can comprise, for example: (1) a first antibody (e.g., attached to a solid support) that binds to a marker protein; and, optionally, (2) a second, different antibody that binds to either the protein or the first antibody and is conjugated to a detectable label.

For oligonucleotide-based kits, the kit can comprise, for example: (1) an oligonucleotide, e.g., a detectably labeled oligonucleotide, which hybridizes to a nucleic acid sequence encoding a marker protein or (2) a pair of primers useful for amplifying a marker nucleic acid molecule. The kit can also comprise, e.g., a buffering agent, a preservative, or a protein stabilizing agent. The kit can further comprise components necessary for detecting the detectable label (e.g., an enzyme or a substrate). The kit can also contain a control sample or a series of control samples which can be assayed and compared to the test sample. Each component of the kit can be enclosed within an individual container and all of the various containers can be within a single package, along with instructions for interpreting the results of the assays performed using the kit.

It is recognized that certain marker proteins are secreted from cervical cells (i.e., one or both of normal and cancerous cells) to the extracellular space surrounding the cells. These markers are preferably used in certain embodiments of the compositions, kits, and methods of the invention, owing to the fact that such marker proteins can be detected in a cervical-associated body fluid sample, which may be more easily collected from a human patient than a tissue biopsy sample. In addition, preferred in vivo techniques for detection of a marker protein include introducing into a subject a labeled antibody directed against the protein. For example, the antibody can be labeled with a radioactive marker whose presence and location in a subject can be detected by standard imaging techniques. An exemplary technique is disclosed in U.S. Pat. No. 6,665,050, hereby incorporated by reference herein.

A preferred agent for detecting marker protein of the invention is an antibody capable of binding to such a protein or a fragment thereof, preferably an antibody with a detectable label. Antibodies can be polyclonal, or more preferably, monoclonal. An intact antibody, or a fragment or derivative thereof (e.g., Fab or F(ab′).sub.2) can be used. In a preferred embodiment, expression of a marker is assessed using a labeled antibody (e.g., a radio-labeled, chromophore-labeled, fluorophore-labeled, or enzyme-labeled antibody), an antibody derivative (e.g., an antibody conjugated with a substrate or with the protein or ligand of a protein-ligand pair such as biotin-streptavidin), or an antibody fragment (e.g., a single-chain antibody, an isolated antibody hypervariable domain, etc.) that binds specifically with a marker protein or fragment thereof, including a marker protein which has undergone all or a portion of its normal post-translational modification.

An exemplary method for detecting the presence or absence of a marker protein or nucleic acid in a biological sample involves obtaining a biological sample (e.g., a cervical-associated body fluid) from a test subject and contacting the biological sample with a compound or an agent capable of detecting the polypeptide or nucleic acid (e.g., mRNA, genomic DNA, or CDNA). The detection methods of the invention can thus be used to detect mRNA, protein, cDNA, or genomic DNA, for example, in a biological sample in vitro as well as in vivo. Exemplary in vitro techniques for detection of mRNA include Northern hybridizations and in situ hybridizations.

Exemplary in vitro techniques for detection of a marker protein include enzyme linked immunosorbent assays (ELISAs), Western blots, immunoprecipitations and immunofluorescence. Exemplary in vitro techniques for detection of genomic DNA include Southern hybridizations. Furthermore, in vivo techniques for detection of a marker protein include introducing into a subject a labeled antibody directed against the protein or fragment thereof. For example, the antibody can be labeled with a radioactive marker whose presence and location in a subject can be detected by standard imaging techniques.

A general principle of such diagnostic and prognostic assays involves preparing a sample or reaction mixture that may contain a marker, and a probe, under appropriate conditions and for a time sufficient to allow the marker and probe to interact and bind, thus forming a complex that can be removed and/or detected in the reaction mixture. These assays can be conducted in a variety of ways.

For example, one method to conduct such an assay would involve anchoring the marker or probe onto a solid phase support, also referred to as a substrate, and detecting target marker/probe complexes anchored on the solid phase at the end of the reaction. In one embodiment of such a method, a sample from a subject, which is to be assayed for presence and/or concentration of marker, can be anchored onto a carrier or solid phase support. In another embodiment, the reverse situation is possible, in which the probe can be anchored to a solid phase and a sample from a subject can be allowed to react as an unanchored component of the assay.

There are many established methods for anchoring assay components to a solid phase. These include, without limitation, marker or probe molecules which are immobilized through conjugation of biotin and streptavidin. Such biotinylated assay components can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemicals). In certain embodiments, the surfaces with immobilized assay components can be prepared in advance and stored.

Other suitable carriers or solid phase supports for such assays include any material capable of binding the class of molecule to which the marker or probe belongs. Well-known supports or carriers include, but are not limited to, glass, polystyrene, nylon, polypropylene, nylon, polyethylene, dextran, amylases, natural and modified celluloses, polyacrylamides, gabbros, and magnetite.

In order to conduct assays with the above mentioned approaches, the non-immobilized component is added to the solid phase upon which the second component is anchored. After the reaction is complete, uncomplexed components may be removed (e.g., by washing) under conditions such that any complexes formed will remain immobilized upon the solid phase. The detection of marker/probe complexes anchored to the solid phase can be accomplished in a number of methods outlined herein. In a preferred embodiment, the probe, when it is the unanchored assay component, can be labeled for the purpose of detection and readout of the assay, either directly or indirectly, with detectable labels discussed herein and which are well-known to one skilled in the art.

It is also possible to directly detect marker/probe complex formation without further manipulation or labeling of either component (marker or probe), for example by utilizing the technique of fluorescence energy transfer (see, for example, Lakowicz et al., U.S. Pat. No. 5,631,169; Stavrianopoulos, et al., U.S. Pat. No. 4,868,103). A fluorophore label on the first, “donor” molecule is selected such that, upon excitation with incident light of appropriate wavelength, its emitted fluorescent energy will be absorbed by a fluorescent label on a second, “acceptor” molecule, which in turn is able to fluoresce due to the absorbed energy. Alternately, the “donor” protein molecule may simply utilize the natural fluorescent energy of tryptophan residues. Labels are chosen that emit different wavelengths of light, such that the “acceptor” molecule label may be differentiated from that of the “donor.” Since the efficiency of energy transfer between the labels is related to the distance separating the molecules, spatial relationships between the molecules can be assessed. In a situation in which binding occurs between the molecules, the fluorescent emission of the “acceptor” molecule label in the assay should be maximal. An FET binding event can be conveniently measured through standard fluorometric detection means well known in the art (e.g., using a fluorimeter).

In another embodiment, determination of the ability of a probe to recognize a marker can be accomplished without labeling either assay component (probe or marker) by utilizing a technology such as real-time Biomolecular Interaction Analysis (BIA) (see, e.g., Sjolander, S. and Urbaniczky, C., 1991, Anal Chem. 63:2338-2345 and Szabo et al., 1995, Curr. Opin. Struct. Biol. 5:699-705). As used herein, “BIA” or “surface plasmon resonance” is a technology for studying biospecific interactions in real time, without labeling any of the interactants (e.g., BIAcore®). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

Alternatively, in another embodiment, analogous diagnostic and prognostic assays can be conducted with marker and probe as solutes in a liquid phase. In such an assay, the complexed marker and probe are separated from uncomplexed components by any of a number of standard techniques, including but not limited to: differential centrifugation, chromatography, electrophoresis and immunoprecipitation. In differential centrifugation, marker/probe complexes may be separated from uncomplexed assay components through a series of centrifugal steps, due to the different sedimentation equilibria of complexes based on their different sizes and densities (see, for example, Rivas, G., and Minton, A. P., 1993, Trends Biochem Sci. 118(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the marker/probe complex as compared to the uncomplexed components may be exploited to differentiate the complex from uncomplexed components, for example through the utilization of ion-exchange chromatography resins. Such resins and chromatographic techniques are well known to one skilled in the art (see, e.g., Heegaard, N. H., 1998, J. Mol. Recognit. 11(1-6):141-8; Hage, D. S., and Tweed, S. A. J Chromatogr B Biomed Sci Appl 1997 699(1-2):499-525). Gel electrophoresis may also be employed to separate complexed assay components from unbound components (see, e.g., Ausubel et al., ed., Current Protocols in Molecular Biology, John Wiley & Sons, New York, 1987-1999). In this technique, protein or nucleic acid complexes are separated based on size or charge, for example. In order to maintain the binding interaction during the electrophoretic process, non-denaturing gel matrix materials and conditions in the absence of reducing agent are typically preferred. Appropriate conditions to the particular assay and components thereof will be well known to one skilled in the art.

In a particular embodiment, the level of marker mRNA can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from cervical cells (see, e.g., Ausubel et al., ed., Current Protocols in Molecular Biology, John Wiley & Sons, New York, 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding a marker of the present invention. Other suitable probes for use in the diagnostic assays of the invention are described herein. Hybridization of an mRNA with the probe indicates that the marker in question is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in, detecting the level of mRNA encoded by the markers of the present invention.

An alternative method for determining the level of mRNA marker in a sample involves the process of nucleic acid amplification, e.g., by rtPCR (the experimental embodiment set forth in Mullis, 1987., U.S. Pat. No. 4,683,202), ligase chain reaction (Barany, 1991, Proc. Natl. Acad. Sci. USA, 88:189-193), self-sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the cervical cells prior to detection. In such methods, a cell or tissue sample is prepared/processed using known histological methods. The sample is then immobilized on a support, typically a glass slide, and then contacted with a probe that can hybridize to mRNA that encodes the marker.

As an alternative to making determinations based on the absolute expression level of the marker, determinations may be based on the normalized expression level of the marker. Expression levels are normalized by correcting the absolute expression level of a marker by comparing its expression to the expression of a gene that is not a marker, e.g., a housekeeping gene that is constitutively expressed. Suitable genes for normalization include housekeeping genes such as the actin gene, or epithelial cell-specific genes. This normalization allows the comparison of the expression level in one sample, e.g., a patient sample, to another sample, e.g., a non-cervical cancer sample, or between samples from different sources.

Alternatively, the expression level can be provided as a relative expression level. To determine a relative expression level of a marker, the level of expression of the marker is determined for 10 or more samples of normal versus high-grade dysplastic cell isolates, preferably 50 or more samples, prior to the determination of the expression level for the sample in question. The mean expression level of each of the genes assayed in the larger number of samples is determined and this is used as a baseline expression level for the marker. The expression level of the marker determined for the test sample (absolute level of expression) is then divided by the mean expression value obtained for that marker. This provides a relative expression level.

Preferably, the samples used in the baseline determination will be from high-grade dysplastic or from non-cervical cancer cells of cervical tissue. The choice of the cell source is dependent on the use of the relative expression level. Using expression found in normal tissues as a mean expression score aids in validating whether the marker assayed is cervical specific (versus normal cells). In addition, as more data is accumulated, the mean expression value can be revised, providing improved relative expression values based on accumulated data. Expression data from cervical dysplastic cells provides a means for grading the severity of the dysplasia.

### Methods

Preliminary Method Evaluation: Prior to the protein discovery work utilizing clinical specimens, several pilot studies were completed to assess the compatibility of PreservCyt® Solution (Cytyc Corporation) and ThinPrep® Pap Stain (Cytyc Corporation) with subsequent sample processing methods and mass spectroscopy analysis. PreservCyt fixative is a proprietary methanol-based buffered preservative solution designed to support cells during transport and slide preparation on the ThinPrep 2000 or 3000 Processor. PreservCyt Solution has routinely been utilized for the collection, storage, and processing of gynecological samples as well as Fine Need Aspirates (FNA), mucoid specimens, body fluids, and superficial brushings and scrapings. The ThinPrep Pap Stain is a specialized cocktail of individual stains (including hematoxylin, Orange G, Eosin) which has been specifically optimized for the visualization and diagnosis of cervical cytology specimens.

An in-solution cell staining protocol was developed to evaluate HeLa cells that had been cultured, harvested at confluency, and placed into PreservCyt solution. Cells in solution were split and subsequently processed through a series of incubation and wash steps utilizing protocols adapted from existing slide-based staining procedures to stain cells with either hematoxylin or the ThinPrep Pap Stain. Because hematoxylin stained tissue has been successfully utilized in mass spectroscopy, protein recovery for cells processed using hematoxylin was directly compared to cells processed with the ThinPrep stain. No significant differences in protein recovery were observed between the two staining methods. Because the ThinPrep Pap stain provides improved morphological discrimination of dysplastic cells proved to be compatible with subsequent mass spectroscopy methods, this stain was utilized in the processing of all cervical specimens.

Sample Procurement Residual ThinPrep cervical specimens having a diagnosis of Within Normal Limits (WNL) or HSIL were evaluated for overall cellularity as well as the percentage of high-grade cells (HSIL specimens). An initial ThinPrep slide (control slide) was prepared on the ThinPrep 2000 instrument from residual clinical samples and subsequently ThinPrep Pap stained and coverslipped. This control slide was utilized to confirm clinical diagnosis and select specimens suitable for inclusion in the study. In addition to requirements of adequate cellularity, all specimens met additional inclusion criteria such as a minimal prevalence of polymorphonuclear neutrophils (PMN's) and bacteria. Finally, selected specimens were processed approximately 6 weeks or less from the date of collection in an effort to minimize potential protein degradation.

For specimens having adequate cellularity, the ThinPrep processor and filter routinely applies approximately 70,000 cells to the slide in a homogeneous thin layer. Because the actual number of high-grade cells can vary substantially between specimens, multiple slides were prepared from selected cases having the highest percentage of dysplastic cells and Pap stained. Abnormal cells from HSIL specimens were identified and marked on the back side of slides using a xylene resistant pen (0.20 mm Pigma pen, Sakura Color Products Corp.) in conjunction with Cytyc's ThinPrep Imaging System Review Scope®. Reference marks served as locator guides for the LCM operator to identify the high-grade cells or cell clusters of interest. A cell count (number of high-grade cells) was performed at this time to ensure that a minimum of 12,000 cells were available per specimen. A total of 116 residual ThinPrep samples having an initial clinical diagnosis of HSIL were acquired and evaluated; of these 10 samples (˜9%) satisfied all inclusion requirements and were prepared for LCM. A total of nine HSIL (abnormal), one AGUS (abnormal) and 13 WNL (normal) samples were analyzed for this study. (AGUS=atypical glandular cells of undetermined significance)

Sample Assessment: To assist in the interpretation of proteomic results, a macroscopic cellular assessment was completed for each WNL and HSIL specimen control slide. Evaluations included estimates for the percentage of superficial, intermediate, and parabasal cells as well as endocervical and metaplastic cellular components. This information was documented for WNL specimens to better understand how potential differences in protein profiles might be attributed to differences in cellular content. For HSIL specimens, patient follow-up information was also requested to permit the segregation of potential patterns. Finally, a 0.25 ml aliquot was removed from each HSIL specimen for HPV genotype analysis using the Roche Linear Array HPV Genotyping Test. A 4 ml aliquot was removed from WNL specimens and subject to analysis using Digene's HCII test for the detection of low-risk or high-risk HPV.

Laser Capture Microdissection (LCM): Coverslips were removed from Pap stained slides with xylene and air-dried. Prior to LCM, Prep Strips were applied to remove poorly adhered material and help reduce overall background. Immediately before cell capture, a drop of xylene was applied to the slide to allow visualization of the cells for coordinate selection. Cycles of xylene application, coordinate selection, and drying were utilized to identify and capture high-grade cells present on the slide. Cells were collected using CapSure polyethylene membrane caps and the LCM caps subsequently placed in eppendorf tubes. Approximately 12,000 cells per specimen were selected using the Autopix LCM System® from Arcturus (Mountain View, Calif.).

Quality control was performed to assess both the background and accuracy of cell removal during the LCM process. This was accomplished by imaging representative areas of the slide before and after LCM. In summary, two slides from each case were selected for quality control and a total of 8 before and after images were taken from each slide (2 images per slide quadrant). Finally, a full image of the LCM cap was taken for all caps. Images were reviewed by a cytotechnologist to quantify the accuracy of selective abnormal cell removal as well as the approximate number of normal cells unintentionally removed (background). Background for the majority of slides was determined to be less than five percent for all samples.

SDS-PAGE: Lysis buffer (2% SDS) was added to the Eppendorf tube to solubilize LCM captured cells. Protein extract was subjected to SDS-PAGE to separate proteins by molecular weight. The gels were divided into three sections and in-gel tryptic digestion performed.
LTQ FT Mass Spectroscopy (MS): Proteolytic samples were analyzed by on-line liquid chromatography using a Thermo Electron linear ion trap with Fourier transfer mass spectrometer (LTQ-FT) with a Dionex nanoLC instrument and a 75 μm ID×15 cm C-18 capillary column (flow rate of 300 mL per minute). Mass spectrometry was performed as 1 full FT-MS scan followed by 8 sequential LTQ-MS/MS scans throughout the 90-minute separation.
Protein Identification and Quantitation: ProteinProphet probability software was utilized first to identify proteins based upon corresponding peptide sequences with >95% confidence, followed by confirmation from accurate mass assignment (within 5 ppm). The peak area from the extract ions (i.e. disease and normal) were used for comparison (differential quantitation).

### Experimental

Method: Cervical specimens were evaluated for overall cellularity as well as the percentage of having a diagnosis of high-grade squamous intraepithelial lesion (HSIL) cells. Multiple slides were prepared from selected cases, and subsequently imaged utilizing Pap stained and ThinPrep Imaging System. Cells selected for LCM were marked using the Review Scope. Approximately 12,000 high-grade cells per specimen were captured via LCM using the Autopix System®. Cells were then lysed with SDS and proteins separated via SDS-PAGE in preparation for in-gel digestion. The resulting peptides were analyzed by on-line liquid chromatography with a LTQ-FTMS. Proteins with different quantitation levels between normal and HSIL samples were identified by comparing the intensities of the representative peptide ions after normalization with intrinsic house keeping proteins and/or cell numbers.
Results: Diagnostic cells of interest from ThinPrep cervical cytology specimens were identified, selected via LCM, and successfully processed for proteomic analysis using mass spectroscopy. To validate this approach, reproducibility and dynamic range were first studied. Less than 30% variation for a given sample was observed for the entire process, and good linearity (r2=0.95) from 3,000 to 24,000 cells was obtained. Following this, 10 disease (HSIL) and 10 normal LCM samples were globally investigated. 2,184 proteins with at least 2 peptide identifications, and including one peptide with accurate mass, a total of 4300 unique proteins were identified. Many proteins were found to be up- or down-regulated with at least a 3-fold difference, particularly in nuclear and mitochondrial regions, based on Gene Ontology software. Due to the sensitivity and dynamic range of this approach, very few cells were required for analysis, and quantitation without labeling was successfully employed. Protein profiles unique to high-grade dysplastic cells can yield potential biomarkers for molecular diagnostic applications.

These results are illustrated in the following Tables.

TABLE 1
Relative Abundant Proteins With Significant Biological Interest Upregulated in
High-Grade Cervical Specimens

| | | Number Up- | |
|---|---|---|---|
| Gene/Protein Name | GI Accession | Regulated Pairs | UniGene ID |
| Heterogeneous nuclear ribonucleoproteins A2/B1 | 133257 | 9 | HS.487774 |
| Heterogeneous nuclear ribonucleoproteins C1/C2 | 20455507 | 9 | HS.508848 |
| Heterogeneous nuclear ribonucleoprotein K | 48429103 | 8 | HS.522257 |
| Heterogenous nuclear ribonucleoprotein U | 6226894 | 7 | HS.166463 |
| Polypyrimidine tract-binding protein 1 | 131528 | 5 | HS.172550 |
| Heterogeneous nuclear ribonucleoprotein A1 | 133254 | 4 | HS.534921, HS.546261 |
| Heterogeneous nuclear ribonucleoprotein F | 1710628 | 4 | HS.808 |
| Heterogeneous nuclear ribonucleoprotein H | 1710632 | 4 | HS.202166 |
| Heterogeneous nuclear ribonucleoprotein R | 12230547 | 4 | HS.373763 |
| Heterogeneous nuclear ribonucleoprotein D0 | 13124489 | 3 | HS.480073 |
| Heterogeneous nuclear ribonucleoprotein M | 55977747 | 3 | HS.465808 |
| Heterogeneous nuclear ribonucleoprotein H | 2500576 | 2 | HS.278857 |
| Heterogeneous nuclear ribonucleoprotein L | 133274 | 1 | HS.446623 |
| Heterogeneous nuclear ribonucleoprotein A3 | 51338779 | 1 | HS.516539 |
| Transgelin-2 | 586000 | 10 | HS.517168 |
| Nucleolin | 128841 | 6 | HS.79110 |
| ADP, ATP carrier protein, fibroblast isoform | 113459 | 9 | HS.522767 |
| Voltage-dependent anion-selective channel protein 2 | 1172554 | 8 | HS.355927 |
| Voltage-dependent anion-selective channel protein 1 | 130683 | 4 | HS.519320 |
| Superoxide dismutase [Mn], mitochondrial precursor | 134665 | 4 | HS.487046 |
| 60S ribosomal protein L7 | 133021 | 6 | HS.421257 |
| 60S acidic ribosomal protein P0 | 133041 | 4 | HS.546285, HS.448 |
| 40S ribosomal protein S5 | 22002064 | 3 | HS.378103 |
| Keratin, type I cytoskeletal 18 | 125083 | 4 | HS.406013 |
| Peptidyl-prolyl cis-trans isomerase A | 51702775 | 4 | HS.356331, HS.517 |
| | | | HS.356331 |
| Peptidyl-prolyl cis-trans isomerase B precursor | 118090 | 5 | HS.434937 |
| GTP-binding nuclear protein Ran | 51338598 | 4 | HS.10842, HS.519656 |
| Ras-related protein Rab-7 | 1709999 | 3 | HS.15738 |
| Heat shock protein HSP 90-alpha | 123678 | 4 | HS.523560, HS.525600 |
| Heat shock protein HSP 90-beta | 17865718 | 5 | HS.509736 |
| Heat shock 70 kDa protein 1 | 462325 | 4 | HS.405994, HS.520028 |
| | | | HS.274402, HS.490287 |
| | | | HS.546245, HS.533257 |
| | | | HS.522463, HS.439552 |
| | | | HS.52 |
| Pyruvate kinase, isozymes M1/M2 | 20178296 | 6 | HS.198281 |
| Ubiquitin-activating enzyme E1 | 24418865 | 3 | HS.533273 |
| Myosin-9 | 6166599 | 3 | HS.474751 |
| SET protein | 46397790 | 3 | HS.436687 |
| Histone H2A.z | 121994 | 4 | HS.119192 |
| T-complex protein 1, beta subunit | 6094436 | 3 | HS.189772 |
| Erythrocyte band 7 integral membrane protein | 114823 | 4 | HS.253903 |
| Moesin | 127234 | 4 | HS.87752 |
| Peroxiredoxin 1 | 548453 | 7 | HS.180909 |
| Sodium/potassium-transporting ATPase alpha-1 chain | 114374 | 3 | HS.371889 |
| precursor | | | |
| Elongation factor Tu, mitochondrial precursor | 1706611 | 3 | HS.12084 |

TABLE 2
Relative Abundant Proteins Upregulated in High-Grade Cervical Specimens

| | | Number Up- | |
|---|---|---|---|
| Gene/Protein Name | GI Accession | Regulated Pairs | UniGene ID |
| Malate dehydrogenase, mitochondrial precursor | 6648067 | 8 | HS.520967 |
| Keratin, type II cytoskeletal 7 | 20178293 | 8 | HS.411501 |
| Vimentin | 55977767 | 8 | HS.533317 |
| Endoplasmin precursor | 119360 | 7 | HS.459507, HS.192 |
| 60 kDa heat shock protein, mitochondrial precursor | 129379 | 7 | HS.471014, HS.113 |
| Transketolase | 1729976 | 7 | HS.89643 |
| Protein disulfide-isomerase A3 precursor | 2507461 | 7 | HS.308709 |
| Stress-70 protein, mitochondrial precursor | 21264428 | 7 | HS.547532, HS.184 |
| Dolichyl-diphosphooligosaccharide-protein | 132559 | 6 | HS.518244 |
| glycosyltransferase 67 kDa subunit precursor | | | |
| Histone H1.2 | 417101 | 6 | HS.7644 |
| Core histone macro-H2A.1 | 12643340 | 6 | HS.420272 |
| Neutrophil defensin 3 precursor | 30316323 | 6 | HS.294176, HS.380 |
| Tubulin beta-1 chain | 56757569 | 6 | HS.294176 |
| 14-3-3 protein tau | 112690 | 5 | HS.74405 |
| Annexin A5 | 113960 | 5 | HS.480653 |
| Apolipoprotein A-I precursor | 113992 | 5 | HS.534983, HS.93194 |
| Calreticulin precursor | 117501 | 5 | HS.515162 |
| Adenylyl cyclase-associated protein 1 | 399184 | 5 | HS.370581 |
| Calnexin precursor | 543920 | 5 | HS.529890 |
| L-plastin | 1346733 | 5 | HS.381099 |
| Annexin A4 | 1703319 | 5 | HS.422986 |
| Annexin A11 | 1703322 | 5 | HS.530291 |
| Keratin, type II cytoskeletal 8 | 2506774 | 5 | HS.533782 |
| Myeloblastin precursor | 6174926 | 5 | HS.928 |
| Nebulin | 19856971 | 5 | HS.134602, HS.529265 |
| Trifunctional enzyme alpha subunit, mitochondrial | 20141376 | 5 | HS.516032 |
| precursor | | | |
| Hemoglobin alpha chain | 57013850 | 5 | |
| Alpha-2-macroglobulin precursor | 112911 | 4 | HS.212838 |
| ATP synthase alpha chain, mitochondrial precursor | 114517 | 4 | HS.298280 |
| Leukocyte elastase precursor | 119292 | 4 | HS.99863 |
| Protein disulfide-isomerase A4 precursor | 119530 | 4 | HS.93659 |
| Haptoglobin precursor | 123508 | 4 | HS.513711 |
| Lamin A/C | 125962 | 4 | HS.491359 |
| Ig mu chain C region | 127514 | 4 | HS.525648 |
| Myeloperoxidase precursor | 129825 | 4 | HS.458272 |
| Fibrinogen beta chain precursor | 399492 | 4 | HS.300774 |
| 40S ribosomal protein S3 | 417719 | 4 | HS.546286 |
| Alpha-1-antitrypsin precursor | 1703025 | 4 | HS.525557 |
| Clathrin heavy chain 1 | 1705916 | 4 | HS.491351 |
| Peroxisomal multifunctional enzyme type 2 | 1706396 | 4 | HS.406861 |
| Probable ATP-dependent RNA helicase p47 | 2500529 | 4 | HS.254042 |
| Fibronectin precursor | 2506872 | 4 | HS.203717 |
| Thioredoxin-dependent peroxide reductase, | 2507171 | 4 | HS.523302 |
| mitochondrial precursor | | | |
| WD-repeat protein 1 | 12643636 | 4 | HS.128548 |
| Epiplakin | 14194713 | 4 | HS.200412 |
| Histone H1.5 | 19856407 | 4 | HS.131956 |
| Isocitrate dehydrogenase [NADP], mitochondrial | 20141568 | 4 | HS.513141 |
| precursor | | | |
| Fibrinogen gamma chain precursor | 20178280 | 4 | HS.546255 |
| Filamin B | 38257363 | 4 | HS.476448 |
| DNA-dependent protein kinase catalytic subunit | 38258929 | 4 | HS.491682 |
| Hook homolog 1 | 41688595 | 4 | HS.378836 |
| Alpha-actinin 1 | 46397817 | 4 | HS.509765 |
| 40S ribosomal protein S8 | 50403622 | 4 | HS.512675 |
| Histone H3.3 | 55977062 | 4 | |
| Tubulin alpha-1 chain | 55977476 | 4 | HS.75318 |
| Hemoglobin beta chain | 56749856 | 4 | HS.523443 |
| Alpha-1-acid glycoprotein 1 precursor | 112877 | 3 | HS.494894 |
| Filamin A | 113001 | 3 | HS.195464 |
| Fructose-bisphosphate aldolase A | 113606 | 3 | HS.513490 |
| Nucleophosmin | 114762 | 3 | HS.519452, HS.535499 |
| | | | HS.196534 |
| Cathepsin G precursor | 115725 | 3 | HS.421724 |
| Complement C3 precursor | 116594 | 3 | HS.529053 |
| Elongation factor 2 | 119172 | 3 | HS.515070 |
| Ig gamma-4 chain C region, Ig gamma-2 chain C region | 121043 | 3 | null |
| Ig gamma-4 chain C region, Ig gamma-2 chain C region | 121047 | 3 | HS.534324 |
| Hemoglobin delta chain | 122713 | 3 | HS.36977 |
| ATP-dependent DNA helicase II, 70 kDa subunit | 125729 | 3 | HS.292493 |
| Ig kappa chain V-III region HAH precursor | 125817 | 3 | |
| Lamin B1 | 125953 | 3 | HS.89497 |
| 40S ribosomal protein SA | 125969 | 3 | HS.374553 |
| Myosin regulatory light chain 2, nonsarcomeric | 127169 | 3 | HS.190086 |
| T-complex protein 1, alpha subunit | 135538 | 3 | HS.487054 |
| Serotransferrin precursor | 136191 | 3 | HS.518267 |
| Thymidine phosphorylase precursor | 136588 | 3 | HS.531314, HS.546 |
| Vitronectin precursor | 139653 | 3 | HS.2257 |
| Zinc-alpha-2-glycoprotein precursor | 141596 | 3 | HS.546239 |
| Prohibitin | 464371 | 3 | HS.514303 |
| Macrophage capping protein | 729022 | 3 | HS.516155 |
| Glutamine synthetase | 1169929 | 3 | HS.518525 |
| Ras GTPase-activating-like protein IQGAP1 | 1170586 | 3 | HS.430551 |
| Nicotinamide phosphoribosyltransferase | 1172027 | 3 | HS.489615 |
| ATP synthase oligomycin sensitivity conferral protein, | 1352049 | 3 | HS.409140 |
| mitochondrial precursor | | | |
| Fructose-1,6-bisphosphatase | 1352403 | 3 | HS.494496 |
| Fibrinogen alpha/alpha-E chain precursor | 1706799 | 3 | HS.351593 |
| Splicing factor, proline-and glutamine-rich | 1709851 | 3 | HS.355934 |
| Calgizzarin | 1710818 | 3 | HS.417004 |
| Protein disulfide-isomerase A6 precursor | 2501205 | 3 | HS.212102, HS.372429 |
| Probable RNA-dependent helicase p72 | 3122595 | 3 | HS.528305 |
| Keratin, type I cuticular HA1 | 6016413 | 3 | HS.41696 |
| Histone H2B.e | 7387742 | 3 | HS.182432 |
| Chloride intracellular channel protein 1 | 12643390 | 3 | HS.414565 |
| Serine protease inhibitor Kazal-type 5 precursor | 13959398 | 3 | HS.331555 |
| Plectin 1 | 14195007 | 3 | HS.434248 |
| Talin 1 | 14916725 | 3 | HS.375001 |
| 78 kDa glucose-regulated protein precursor | 14916999 | 3 | HS.522394 |
| Crumbs protein homolog 1 precursor | 17374421 | 3 | HS.126135 |
| Ryanodine receptor 3 | 18202506 | 3 | HS.445841 |
| Probable ATP-dependent helicase DDX48 | 20532400 | 3 | HS.389649 |
| Isocitrate dehydrogenase [NADP] cytoplasmic | 21903432 | 3 | HS.11223 |
| Vinculin | 21903479 | 3 | HS.500101 |
| 60S ribosomal protein L4 | 22002063 | 3 | HS.186350, HS.4328 |
| Fibulin-1 precursor | 30581038 | 3 | HS.24601 |
| Ras-related protein Rab-11B | 38258938 | 3 | HS.433888 |
| Dolichyl-diphosphooligosaccharide-Protein | 46397832 | 3 | HS.523145 |
| glycosyltransferase 48 kDa subunit precursor | | | |
| Lysozyme C precursor | 48428995 | 3 | HS.524579 |
| Keratin, type II cuticular HB5 | 48474780 | 3 | HS.182507 |
| Nuclear mitotic apparatus protein 1 | 50400858 | 3 | HS.523873 |
| 40S ribosomal protein S4, X isoform | 50403628 | 3 | HS.446628 |
| T-complex protein 1, delta subunit | 52001478 | 3 | HS.421509 |
| Tubulin alpha-ubiquitous chain | 55977474 | 3 | HS.524390 |
| Citrate synthase, mitochondrial precursor | 57015285 | 3 | |

TABLE 3
Lower Probability Proteins Upregulated in High-Grade Cervical Specimens

| | | Number Up- | |
|---|---|---|---|
| Gene/Protein Name | GI Accession | Regulated Pairs | UniGene ID |
| Alpha-1-antichymotrypsin precursor | 112874 | 2 | HS.534293 |
| Aspartate aminotransferase, mitochondrial precursor | 112983 | 2 | HS.460929 |
| Ig alpha-1 chain C region | 113584 | 2 | null |
| Alcohol dehydrogenase [NADP+] | 113600 | 2 | HS.474584 |
| Antithrombin-III precursor | 113936 | 2 | HS.75599 |
| Annexin A3 | 113954 | 2 | HS.480042 |
| Annexin A6 | 113962 | 2 | HS.412117 |
| Annexin A8 | 113967 | 2 | HS.463110, HS.524 |
| Apolipoprotein B-100 precursor | 114014 | 2 | HS.120759 |
| ATP synthase beta chain, mitochondrial precursor | 114549 | 2 | HS.406510 |
| Carbonic anhydrase II | 115456 | 2 | HS.155097 |
| Calpain small subunit 1 | 115612 | 2 | HS.515371 |
| Ceruloplasmin precursor | 116117 | 2 | HS.282557 |
| Clusterin precursor | 116533 | 2 | HS.436657 |
| Alpha enolase | 119339 | 2 | HS.517145 |
| Ferritin light chain | 120523 | 2 | HS.433670 |
| Guanine nucleotide-binding protein G(i), alpha-2 subunit | 121023 | 2 | HS.77269 |
| Ig gamma-1 chain C region | 121039 | 2 | HS.375600 |
| Solute carrier family 2, facilitated glucose transporter, | 121751 | 2 | HS.473721 |
| member 1 | | | |
| Histone H1.3 | 121925 | 2 | HS.136857 |
| Histone H2A.o | 121970 | 2 | HS.530461 |
| Histone H2A.x | 121992 | 2 | HS.477879 |
| Ig heavy chain V-I region HG3 precursor | 123799 | 2 | |
| Ig heavy chain V-III region TEI, Ig heavy chain V-III | 123845 | 2 | |
| region BRO | | | |
| Keratin, type I cytoskeletal 15 | 125081 | 2 | HS.80342 |
| ATP-dependent DNA helicase II, 80 kDa subunit | 125731 | 2 | HS.388739 |
| Leukotriene A-4 hydrolase | 126353 | 2 | HS.524648 |
| Galectin-3 | 126678 | 2 | HS.531081 |
| Tyrosine-protein phosphatase, non-receptor type 6 | 131469 | 2 | HS.63489 |
| Prolactin-inducible protein precursor | 134170 | 2 | HS.99949 |
| Transferrin receptor protein 1 | 136378 | 2 | HS.529618 |
| Transthyretin precursor | 136464 | 2 | HS.427202 |
| Vitamin D-binding protein precursor | 139641 | 2 | HS.418497 |
| Fatty acid-binding protein, epidermal | 232081 | 2 | HS.408061 |
| Tumor necrosis factor, alpha-induced protein 2 | 416700 | 2 | HS.525607 |
| 60S ribasomal protein L9 | 417677 | 2 | HS.412370, HS.513083 |
| Fibrillin 1 precursor | 544279 | 2 | HS.146447 |
| Keratin, type I cytoskeletal 17 | 547751 | 2 | HS.2785 |
| Serine/threonine protein phosphatase PP1-gamma | 548573 | 2 | HS.79081 |
| catalytic subunit | | | |
| Myeloid cell nuclear differentiation antigen | 730038 | 2 | HS.153837 |
| UTP-glucose-1-phosphate uridylyltransferase 1 | 731050 | 2 | null |
| Neutrophil gelatinase-associated lipocalin precursor | 1171700 | 2 | HS.204238 |
| 14-3-3 protein beta/alpha | 1345590 | 2 | HS.279920 |
| 60S ribosomal protein L6 | 1350762 | 2 | HS.546283, HS.5286 |
| Phosphatidylethanolamine-binding protein | 1352726 | 2 | HS.433863 |
| F-actin capping protein alpha-1 subunit | 1705650 | 2 | HS.514934 |
| Coatomer alpha subunit | 1705996 | 2 | HS.162121 |
| Hemopexin precursor | 1708182 | 2 | HS.426485 |
| Hexokinase type III | 1708363 | 2 | HS.411695 |
| Malate dehydrogenase, cytoplasmic | 1708967 | 2 | HS.526521 |
| 130 kDa leucine-rich protein | 1730078 | 2 | HS.368084 |
| 3-hydroxyacyl-CoA dehydrogenase type II | 2492759 | 2 | HS.171280 |
| Laminin alpha-2 chain precursor | 2506805 | 2 | HS.200841 |
| Protein disulfide-isomerase precursor | 2507460 | 2 | HS.464336 |
| Pyridoxal kinase | 2811007 | 2 | HS.284491 |
| Enoyl-CoA hydratase, mitochondrial precursor | 2851395 | 2 | HS.76394 |
| DEAD-box protein 3, X-chromosomal | 3023628 | 2 | HS.380774 |
| Actin-related protein ⅔ complex subunit 2 | 3121764 | 2 | HS.529303 |
| 2,4-dienoyl-CoA reductase, mitochondrial precursor | 3913456 | 2 | HS.492212 |
| ATP-dependent RNA helicase A | 3915658 | 2 | HS.191518 |
| Carcinoembryonic antigen-related cell adhesion | 5921734 | 2 | HS.74466 |
| molecule 7 precursor | | | |
| Lactotransferrin precursor | 6175096 | 2 | HS.529517 |
| Transaldolase | 6648092 | 2 | HS.438678 |
| Eukaryotic translation initiation factor 3 subunit 10 | 6685537 | 2 | HS.523299 |
| Keratin, type I cuticular HA6 | 6685565 | 2 | HS.248189 |
| Aconitate hydratase, mitochondrial precursor | 6686275 | 2 | HS.474982 |
| Hsc70-interacting protein | 6686278 | 2 | HS.546303 |
| Keratin, type I cuticular HA5 | 6686303 | 2 | HS.73082 |
| Poly(rC)-binding protein 2 | 6707736 | 2 | HS.546271 |
| Dolichyl-diphosphooligosaccharide-protein | 9297108 | 2 | HS.370895 |
| glycosyltransferase 63 kDa subunit precursor | | | |
| 6-phosphofructokinase, liver type | 9988057 | 2 | HS.255093 |
| T-complex protein 1, theta subunit | 9988062 | 2 | HS.125113 |
| Coatomer gamma subunit | 12229771 | 2 | HS.518250 |
| Zinc finger protein 208 | 12585543 | 2 | HS.419763 |
| Proteasome subunit alpha type 7 | 12643540 | 2 | HS.233952 |
| Myeloid/lymphoid or mixed-lineage leukemia protein 4 | 12643900 | 2 | HS.92236 |
| 54 kDa nuclear RNA- and DNA-binding protein | 13124797 | 2 | HS.533282 |
| Catenin delta-1 | 14916543 | 2 | HS.166011 |
| Growth hormone inducible transmembrane protein | 15213977 | 2 | HS.352656 |
| Glucose-6-phosphate isomerase | 17380385 | 2 | HS.466471 |
| Proteasome activator complex subunit 2 | 18203506 | 2 | HS.512410, HS.434081 |
| EF-hand domain-containing protein 2 | 20140139 | 2 | HS.465374 |
| Complement C4 precursor | 20141171 | 2 | HS.534847, HS.546241 |
| | | | HS.534847, HS.546241 |
| Argininosuccinate synthase | 20141195 | 2 | HS.160786 |
| Collagen-binding protein 2 precursor | 20141241 | 2 | HS.241579 |
| Dermcidin precursor | 20141302 | 2 | HS.350570 |
| Tubulin alpha-6 chain | 20455322 | 2 | HS.436035 |
| ATP synthase B chain, mitochondrial precursor | 20455474 | 2 | HS.514870 |
| Carcinoembryonic antigen-related cell adhesion | 20455477 | 2 | HS.466814 |
| molecule 6 precursor | | | |
| 26S proteasome non-ATPase regulatory subunit 3 | 20532405 | 2 | HS.12970 |
| Importin beta-1 subunit | 20981701 | 2 | HS.532793 |
| D-3-phosphoglycerate dehydrogenase | 21264510 | 2 | HS.487296 |
| Major vault protein | 21542417 | 2 | HS.513488 |
| Niban-like protein | 22256935 | 2 | HS.522401 |
| Cytosolic nonspecific dipeptidase | 23396498 | 2 | HS.149185 |
| Normal mucosa of esophagus specific gene 1 protein | 23396774 | 2 | HS.112242 |
| Myosin If (Myosin-IE) | 23831195 | 2 | HS.408451 |
| N-acetylglucosamine kinase | 24638065 | 2 | HS.7036 |
| Aldehyde dehydrogenase family 7 member A1 | 25108887 | 2 | HS.483239 |
| Myosin XVIIIB | 32699565 | 2 | HS.417959 |
| Myosin VI | 33860183 | 2 | HS.149387 |
| Ras-related protein Rab-5C | 38258923 | 2 | HS.514182 |
| ARP ⅔ complex 20 kDa subunit | 38372625 | 2 | HS.323342 |
| Neuroblast differentiation associated protein AHNAK | 39932547 | 2 | HS.502756 |
| Cytochrome c | 42560196 | 2 | HS.437060 |
| Poly(rC)-binding protein 1 | 42560548 | 2 | HS.2853 |
| Eukaryotic initiation factor 4A-I | 46397463 | 2 | HS.129673 |
| Keratin, type II cuticular HB1 | 46397468 | 2 | HS.185568 |
| 40S ribosomal protein S20 | 46397703 | 2 | HS.8102 |
| Actin-like protein 3 | 47117647 | 2 | HS.433512 |
| 10 kDa heat shock protein, mitochondrial | 47606335 | 2 | HS.1197 |
| Myosin light polypeptide 6 | 47606436 | 2 | HS.505705 |
| 14-3-3 protein gamma | 48428721 | 2 | HS.520974 |
| Keratin, type II cuticular HB6 | 48474260 | 2 | HS.278658 |
| Keratin, type II cuticular HB2 | 48474984 | 2 | HS.134640 |
| 40S ribosomal protein S16 | 50403607 | 2 | HS.397609 |
| 40S ribosomal protein S13 | 50403608 | 2 | HS.446588 |
| 40S ribosomal protein S14 | 50403752 | 2 | H3.381126 |
| Ciliary dynein heavy chain 5 | 51316044 | 2 | HS.520106, HS.212 |
| Histone H4 | 51317339 | 2 | |
| Ras-related protein Rab-1A | 51338603 | 2 | HS.310645 |
| Small nuclear ribonucleoprotein Sm D1 | 51338665 | 2 | HS.464734 |
| 14-3-3 protein epsilon | 51702210 | 2 | HS.513851 |
| 60S ribosomal protein L30 | 51702805 | 2 | HS.400295 |
| Phosphoglycerate kinase 1 | 52788229 | 2 | HS.78771 |
| Guanine nucleotide-binding protein beta subunit 2-like 1 | 54037168 | 2 | HS.5662 |
| Tropomyosin alpha 4 chain | 54039751 | 2 | HS.466088 |
| Tubulin beta-2 chain | 56757569 | 2 | |
| Spectrin alpha chain, brain | 56757656 | 2 | |
| Protein-glutamine gamma-glutamyltransferase K | 57015359 | 2 | |
| Staphylococcal nuclease domain containing protein 1 | 60415926 | 2 | |
| Interleukin enhancer-binding factor 2 | 62510764 | 2 | |
| Probable ubiquitin ligase protein MYCBP2 | 68052838 | 2 | |
| Mannose-6-phosphate receptor binding protein 1 | 68846601 | 2 | |
| Erythrocyte membrane protein band 4.2 | 112798 | 1 | HS.368642 |
| 4F2 cell-surface antigen heavy chain | 112803 | 1 | HS.502769 |
| 5′-nucleotidase precursor | 112825 | 1 | HS.153952 |
| Alpha-2-antiplasmin precursor | 112907 | 1 | HS.159509 |
| ADP/ATP translocase 3 | 113463 | 1 | HS.246506, HS.350 |
| | | | HS.246506 |
| Serum albumin precursor | 113576 | 1 | HS.418167 |
| Fructose-bisphosphate aldolase C | 113613 | 1 | HS.155247 |
| Angiotensinogen precursor | 113880 | 1 | HS.19383 |
| Annexin A1 | 113944 | 1 | HS.494173 |
| Amine oxidase | 113978 | 1 | HS.183109 |
| Apolipoprotein A-II precursor | 114000 | 1 | HS.237658 |
| Apolipoprotein A-IV precursor | 114006 | 1 | HS.1247 |
| Sarcoplasmic/endoplasmic reticulum calcium ATPase 2 | 114312 | 1 | HS.506759 |
| Band 3 anion transport protein | 114787 | 1 | HS443948 |
| Carbonic anhydrase I | 115449 | 1 | HS.23118 |
| Calpain 1, large [catalytic] subunit | 115574 | 1 | HS.502842, HS.492718 |
| Cathepsin D precursor | 115717 | 1 | HS.546248, HS.121575 |
| Carcinoembryonic antigen-related cell adhesion | 115940 | 1 | HS.220529 |
| molecule 5 Precursor | | | |
| Complement factor H precursor | 116131 | 1 | HS.2637, HS.363396 |
| Calcyclin | 116509 | 1 | HS.275243 |
| Complement C5 precursor | 116607 | 1 | HS.494997 |
| Neutrophil collagenase precursor | 116862 | 1 | HS.161839 |
| Matrix metalloproteinase-9 precursor | 116863 | 1 | HS.297413 |
| Cytochrome c oxidase subunit IV isoform 1, | 117086 | 1 | HS.433419 |
| Mitochondrial precursor | | | |
| Cytochrome P450 1A1 | 117139 | 1 | HS.72912 |
| Cytochrome P450 3A7 | 117159 | 1 | HS.111944 |
| Cystatin A | 118177 | 1 | HS.518198 |
| Aldehyde dehydrogenase, mitochondrial precursor | 118504 | 1 | HS.436437 |
| Glutamate dehydrogenase 1, mitochondrial precursor | 118541 | 1 | HS.500409 |
| NAD(P)H dehydrogenase [quinone] 1 | 118607 | 1 | HS.406515 |
| Elongation factor 1-gamma | 119165 | 1 | HS.144835, HS.444467 |
| Eosinophil granule major basic protein precursor | 119239 | 1 | HS.512633 |
| Electron transfer flavoprotein alpha-subunit, | 119636 | 1 | HS.39925 |
| Mitochondrial precursor | | | |
| Ferritin heavy chain | 120516 | 1 | HS.446345, HS.533597 |
| | | | HS.448738, HS.500761 |
| | | | HS.523854 |
| Glucosidase II beta subunit precursor | 120629 | 1 | HS.512640 |
| Gelsolin precursor | 121116 | 1 | HS.522373 |
| Histone H2A.a | 121968 | 1 | HS.121017, HS.2481 |
| HLA class I histocompatibility antigen, A-2 alpha chain | 122138 | 1 | |
| precursor | | | |
| Heparin cofactor II precursor | 123055 | 1 | HS.474270 |
| Ig heavy chain V-II region NEVVM, Ig heavy chain V-II | 123828 | 1 | |
| Region ARH-77 precursor | | | |
| Ig heavy chain V-III region VH26 precursor | 123843 | 1 | null |
| Ig heavy chain V-III region TIL | 123844 | 1 | |
| Ig heavy chain V-III region HIL | 123850 | 1 | |
| Targeting protein for Xklp2 | 124096 | 1 | HS.384598 |
| Insulin-like growth factor I receptor precursor | 124240 | 1 | HS.20573 |
| Integrin beta-2 precursor | 124966 | 1 | HS.375957 |
| Inter-alpha-trypsin inhibitor heavy chain H2 precursor | 125000 | 1 | HS.75285 |
| Ig kappa chain C region | 125145 | 1 | HS.449621 |
| Creatine kinase, ubiquitous mitochondrial precursor | 125315 | 1 | HS.425633 |
| Ig kappa chain V-III region SIE | 125819 | 1 | |
| Ig kappa chain V-IV region JI precursor, Ig kappa | 125833 | 1 | |
| chain V-IV region precursor | | | |
| L-lactate dehydrogenase B chain | 126041 | 1 | HS.446149 |
| Laminin alpha-1 chain precursor | 126363 | 1 | HS.270364 |
| Neutrophil cytosol factor 1 | 127946 | 1 | HS.458275, HS.1583 |
| | | | HS.448231 |
| Probable RNA-dependent helicase p68 | 129383 | 1 | HS.279806 |
| Junction plakoglobin | 130257 | 1 | HS.514174 |
| Plasminogen precursor | 130316 | 1 | HS.143436 |
| Phosphoglycerate mutase 1 | 130348 | 1 | HS.447492, HS.502841 |
| | | | HS.511830 |
| Poly [ADP-ribose] polymerase-1 | 130781 | 1 | HS.177766 |
| Proteasome subunit alpha type 2 | 130850 | 1 | HS.333786 |
| Placental ribonuclease inhibitor | 132573 | 1 | HS.530687 |
| 60S acidic ribosomal protein P1 | 133051 | 1 | HS.356502 |
| Proactivator polypeptide precursor | 134218 | 1 | HS.523004 |
| Tryptophanyl-tRNA synthetase | 135191 | 1 | HS.497599 |
| Thrombospondin-1 precursor | 135717 | 1 | HS.164226 |
| Acetyl-CoA acetyltransferase, mitochondrial precursor | 135755 | 1 | HS.232375 |
| Thioredoxin | 135773 | 1 | HS.435136 |
| Tumor necrosis factor receptor superfamily member 1A | 135959 | 1 | HS.279594 |
| precursor | | | |
| Tropomyosin alpha 3 chain | 136085 | 1 | HS.406293, HS.146070 |
| | | | HS.546881, HS.449194 |
| | | | HS.518123 |
| 60S ribosomal protein L12 | 266921 | 1 | HS.408054 |
| 14-3-3 protein sigma | 398953 | 1 | HS.523718 |
| Sodium channel protein type VII alpha subunit | 399254 | 1 | HS.182889 |
| FK506-binding protein 4 | 399866 | 1 | HS.524183 |
| S100 calcium-binding protein A7 | 400892 | 1 | HS.112408 |
| Von Willebrand factor precursor | 401413 | 1 | HS.440848 |
| C4b-binding protein alpha chain precursor | 416733 | 1 | HS.1012 |
| Azurocidin precursor | 416746 | 1 | HS.72885 |
| Long-chain-fatty-acid-CoA ligase 1 | 417241 | 1 | HS.406678 |
| Beta-catenin | 461854 | 1 | HS.476018 |
| Metallothionein-IK | 462636 | 1 | HS.433391, HS.188518 |
| ATP synthase gamma chain, mitochondrial precursor | 543875 | 1 | HS.271135 |
| 60S ribosomal protein L18 | 548749 | 1 | HS.515517 |
| Antigen peptide transporter 1 | 549042 | 1 | HS.352018 |
| Complement factor B precursor | 584908 | 1 | HS.69771 |
| Trichohyalin | 586120 | 1 | null |
| Alu subfamily SX sequence contamination warning entry | 728838 | 1 | |
| Peroxisomal farnesylated protein | 729723 | 1 | HS.517232 |
| Proteasome subunit beta type 10 precursor | 730376 | 1 | HS.9661 |
| 40S ribosomal protein S19 | 730640 | 1 | HS.438429 |
| T-complex protein 1, zeta subunit | 730922 | 1 | HS.82916 |
| Ubiquitin carboxyl-terminal hydrolase 8 | 731046 | 1 | HS.443731 |
| Succinate dehydrogenase [ubiquinone] flavoprotein | 1169337 | 1 | HS.440475 |
| subunit, mitochondrial precursor | | | |
| FKBP12-rapamycin complex-associated protein | 1169735 | 1 | HS.338207 |
| Grancalcin | 1170014 | 1 | HS.377894 |
| Glutathione S-transferase Mu 4 | 1170096 | 1 | HS.348387 |
| Proteasome activator complex subunit 1 | 1170519 | 1 | HS.75348 |
| Phosphatidylinositol-4,5-bisphosphate 3-kinase catalytic | 1171953 | 1 | HS.518316 |
| subunit, alpha isoform | | | |
| 40S ribosomal protein S10 | 1173177 | 1 | HS.406620, HS.5391 |
| CENP-F kinetochore protein | 1345731 | 1 | HS.497741 |
| Fibrillin 2 precursor | 1345961 | 1 | HS.519294 |
| Keratin, type II cytoskeletal 1 | 1346343 | 1 | HS.80828 |
| Keratin, type II cytoskeletal 6A | 1346344 | 1 | HS.367762 |
| Keratin, type II cytoskeletal 6B | 1346345 | 1 | HS.524438 |
| Keratin, type II cytoskeletal 6C | 1346346 | 1 | HS.367762 |
| Diacylglycerol kinase, gamma | 1346372 | 1 | HS.171499 |
| Galectin-7 | 1346431 | 1 | HS.99923 |
| Retinoic acid receptor RXR-gamma | 1350913 | 1 | HS.26550 |
| Tissue factor pathway inhibitor 2 precursor | 1351226 | 1 | HS.438231 |
| Complement component C9 precursor | 1352108 | 1 | HS.1290 |
| Eosinophil peroxidase precursor | 1352738 | 1 | HS.279259 |
| Acyl-CoA dehydrogenase, very-long-chain specific, | 1703068 | 1 | HS.437178 |
| Mitochondrial precursor | | | |
| Adapter-related protein complex 1 beta 1 subunit | 1703167 | 1 | HS.368794 |
| Flavin reductase | 1706870 | 1 | HS.515785 |
| Rho GDP-dissociation inhibitor 1 | 1707892 | 1 | HS.159161 |
| Rho GDP-dissociation inhibitor 2 | 1707893 | 1 | HS.504877 |
| Krueppel-related zinc finger protein 3 | 1708212 | 1 | HS.502330 |
| Hydroxymethylglutaryl-CoA synthase, mitochondrial | 1708234 | 1 | HS.59889 |
| precursor | | | |
| Heat shock-related 70 kDa protein 2 | 1708307 | 1 | HS.432648 |
| Serpin B8 | 1709895 | 1 | HS.368077 |
| Regulator of G-protein signaling 3 | 1710136 | 1 | HS.494875 |
| Squamous cell carcinoma antigen 2 | 1710877 | 1 | HS.227948, HS.123035 |
| Translocon-associated protein, delta subunit precursor | 1711550 | 1 | HS.409223 |
| Vascular endothelial growth factor receptor 3 precursor | 1718189 | 1 | HS.415048 |
| Tetratricopeptide repeat protein 3 | 1730008 | 1 | HS.368214 |
| Nucleoprotein TPR | 1730009 | 1 | HS.279640 |
| Ketohexokinase | 1730044 | 1 | HS.159525 |
| Glycine amidinotransferase, mitochondrial precursor | 1730201 | 1 | HS.75335 |
| Polymeric-immunoglobulin receptor precursor | 1730570 | 1 | HS.497589 |
| Desmoglein-2 precursor | 2493421 | 1 | HS.412597 |
| Spectrin beta chain, brain 1 | 2493434 | 1 | HS.503178 |
| Calcyphosine | 2493439 | 1 | HS.26685 |
| I-plastin | 2493466 | 1 | HS.203637 |
| Delta3,5-delta2,4-dienoyl-CoA isomerase, mitochondrial | 2494238 | 1 | HS.196176 |
| precursor | | | |
| Fascin | 2498357 | 1 | HS.118400 |
| Spliceosome RNA helicase BAT1 | 2500529 | 1 | |
| Septin-2 | 2500769 | 1 | HS.335057 |
| Vesicle-associated membrane protein 3 | 2501082 | 1 | HS.66708 |
| Zinc finger protein 239 | 2501707 | 1 | HS.25040 |
| Peroxiredoxin 2 | 2507169 | 1 | HS.432121 |
| CD44 antigen precursor | 2507241 | 1 | HS.502328 |
| NADPH--cytochrome P450 reductase | 2851393 | 1 | HS.354056 |
| Myosin heavy chain, skeletal muscle, perinatal | 3041707 | 1 | HS.534028 |
| Myosin heavy chain, fast skeletal muscle, embryonic | 3043372 | 1 | HS.440895 |
| Actin-related protein ⅔ complex subunit 1B | 3121763 | 1 | HS.489284 |
| Actin-related protein ⅔ complex subunit 3 | 3121765 | 1 | HS.524741 |
| ARP2/3 complex 16 kDa subunit | 3121767 | 1 | HS.518609 |
| Dihydropyrimidinase-related protein 2 | 3122051 | 1 | HS.173381 |
| Type I inositol-1,4,5-trisphosphate 5-phosphatase | 3122245 | 1 | HS.523360 |
| Thiosulfate sulfurtransferase | 3122965 | 1 | HS.474783 |
| Collagen alpha 1(XII) chain precursor | 3182940 | 1 | HS.101302 |
| Protein tyrosine kinase 2 beta | 3183003 | 1 | HS.491322 |
| Polyadenylate-binding protein 1 | 3183544 | 1 | HS.387804 |
| Dehydrogenase/reductase SDR family member 2 | 3915733 | 1 | HS.272499 |
| Transformer-2 protein homolog | 4033480 | 1 | HS.445652 |
| Sorbitol dehydrogenase | 4033691 | 1 | HS.878 |
| Serine/threonine protein phosphatase 2A, 65 kDa | 5915686 | 1 | HS.546276 |
| regulatory subunit A, alpha isoform | | | |
| Collagen alpha 3(VI) chain precursor | 5921193 | 1 | HS.233240 |
| Endoplasmic reticulum protein ERp29 precursor | 6015110 | 1 | HS.75841 |
| Glutathione S-transferase A4-4 | 6016167 | 1 | HS.485557 |
| Ig lambda chain V-III region LOI | 6016518 | 1 | |
| Phytanoyl-CoA dioxygenase, peroxisomal precursor | 6093646 | 1 | HS.498732 |
| Procollagen-lysine, 2-oxoglutarate 5-dioxygenase 2 | 6093730 | 1 | HS.477866 |
| precursor | | | |
| Selenium-binding protein 1 | 6094240 | 1 | HS.334841 |
| Transitional endoplasmic reticulum ATPase | 6094447 | 1 | HS.529782 |
| Voltage-dependent P/Q-type calcium channel alpha-1A | 6166047 | 1 | HS.408449 |
| subunit | | | |
| Dimethylaniline monooxygenase | 6166183 | 1 | HS.445350 |
| NDRG1 protein | 6166568 | 1 | HS.372914 |
| Gamma-synuclein | 6175048 | 1 | HS.349470 |
| Tripeptidyl-peptidase I precursor | 6175068 | 1 | HS.523454 |
| UDP-glucose 6-dehydrogenase | 6175086 | 1 | HS.28309 |
| Heat shock 70 kDa protein 4 | 6226869 | 1 | HS.90093 |
| Membrane associated progesterone receptor | 6647589 | 1 | HS.90061 |
| component 1 | | | |
| Calcineurin-binding protein Cabin 1 | 6685261 | 1 | HS.517478 |
| Claudin-4 | 6685272 | 1 | HS.520942 |
| Keratin, type I cuticular HA4 | 6685564 | 1 | HS.296942 |
| TNF receptor associated factor 3 | 6686035 | 1 | HS.510528 |
| Microsomal glutathione S-transferase 3 | 7387731 | 1 | HS.191734 |
| Histone H2B.q | 7387736 | 1 | HS.2178 |
| Histone H2B.q | 7404367 | 1 | |
| NK-tumor recognition protein | 8039798 | 1 | HS.529509 |
| Forkhead box protein O3A | 8134467 | 1 | HS.220950 |
| Mannose-6-phosphate receptor binding protein 1 | 8134735 | 1 | HS.140452 |
| Acid ceramidase precursor | 8247915 | 1 | HS.527412 |
| Regulator of G-protein signaling 9 | 8475983 | 1 | HS.132327 |
| Tenascin X precursor | 9087217 | 1 | HS.42853, HS.485104 |
| Histone H2B.e | 9973351 | 1 | |
| U6 snRNA-associated Sm-like protein LSm2 | 10720079 | 1 | HS.103106 |
| 150 kDa oxygen-regulated protein precursor | 10720185 | 1 | HS.277704 |
| Zinc finger protein 221 | 11136126 | 1 | HS.274445 |
| Programmed cell death protein 6 | 12230420 | 1 | HS.50823, HS.379186 |
| Zinc finger protein 43 | 12585553 | 1 | HS.534365 |
| Vacuolar ATP synthase subunit B, brain isoform | 12643271 | 1 | HS.295917 |
| Absent in melanoma 1 protein | 12643308 | 1 | HS.486074 |
| Cathepsin Z precursor | 12643324 | 1 | HS.252549 |
| Glutathione S-transferase kappa 1 | 12643338 | 1 | HS.390667 |
| Sorting nexin-3 | 12643620 | 1 | HS.12102 |
| Vacuolar proton translocating ATPase 116 kDa subunit | 12643719 | 1 | HS.495985 |
| A isoform 3 | | | |
| Gamma-aminobutyric acid type B receptor, subunit 1 | 12643873 | 1 | HS.167017 |
| precursor | | | |
| Coronin-1C | 12643898 | 1 | HS.330384 |
| Voltage-dependent anion-selective channel protein 3 | 12643945 | 1 | HS.491597 |
| Desmoplakin | 12644130 | 1 | HS.519873 |
| Probable DNA dC-)dU editing enzyme APOBEC-3A | 12644206 | 1 | HS.348983 |
| Diacylglycerol kinase, zeta | 12644407 | 1 | HS.502461 |
| Myosin light chain kinase, smooth muscle and non- | 12644418 | 1 | HS.477375 |
| Muscle isozymes | | | |
| Coatomer gamma subunit | 13124090 | 1 | HS.532231 |
| Targeting protein for Xklp2 | 13124096 | 1 | HS.244580 |
| F-box only protein 3 | 13124239 | 1 | HS.406787 |
| Long-chain-fatty-acid-CoA ligase 5 | 13431659 | 1 | HS.11638 |
| Myosin Ic | 13431674 | 1 | HS.286226 |
| Keratin, type I cytoskeletal 14 | 13432173 | 1 | HS.355214 |
| Myosin-11 | 13432177 | 1 | HS.460109 |
| Inter-alpha-trypsin inhibitor heavy chain H4 precursor | 13432192 | 1 | HS.518000 |
| Serine-protein kinase ATM | 13878337 | 1 | HS.435561 |
| Lysyl oxidase homolog 2 precursor | 13878585 | 1 | HS.116479 |
| Collagen alpha 1(VI) chain precursor | 13878903 | 1 | HS.474053 |
| Envoplakin | 14194715 | 1 | HS.500635 |
| Vinexin | 14423996 | 1 | HS.528572 |
| Myosin Ixb | 14548118 | 1 | HS.123198 |
| Ras-GTPase-activating protein binding protein 2 | 14916573 | 1 | HS.303676 |
| Glutathione reductase, mitochondrial precursor | 14916998 | 1 | HS.271510 |
| Splicing factor 3B subunit 1 | 15214275 | 1 | HS.471011 |
| ADAMTS-12 precursor | 17366354 | 1 | HS.481865 |
| Inositol 1,4,5-trisphosphate receptor type 3 | 17366458 | 1 | HS.65758 |
| Spectrin beta chain, brain 2 | 17367904 | 1 | HS.26915 |
| Reticulon 4 | 17369290 | 1 | HS.429581 |
| Ryanodine receptor 2 | 17380312 | 1 | HS.109514 |
| Transcriptional regulator ATRX | 17380440 | 1 | HS.533526 |
| Acyl-coenzyme A oxidase 1, peroxisomal | 17380467 | 1 | HS.464137 |
| Heat shock protein 75 kDa, mitochondrial precursor | 17865679 | 1 | HS.30345 |
| Histone H3/b, Histone H3.1 | 18202621 | 1 | HS.546315, HS.532 |
| | | | HS.248176, HS.44 |
| | | | HS.132854, HS.24 |
| | | | HS.484990, HS.70 |
| | | | HS.533292, HS.24 |
| Tripartite motif protein 8 | 18202744 | 1 | HS.336810 |
| Echinoderm microtubule-associated protein-like 4 | 18202954 | 1 | HS.432438 |
| PINCH protein | 18266876 | 1 | HS.469593 |
| Heat-shock protein beta-1 | 19855073 | 1 | HS.520973 |
| Ryanodine receptor 1 | 19857096 | 1 | HS.466664 |
| DNA replication licensing factor MCM5 | 19858646 | 1 | HS.517582 |
| Zinc finger protein 268 | 19863363 | 1 | HS.183291, HS.186387 |
| Cytochrome P450 3A43, Cytochrome P450 3A3 | 20137481 | 1 | HS.306220 |
| Interleukin-4 induced protein 1 precursor | 20138284 | 1 | HS.467133 |
| Methylcrotonoyl-CoA carboxylase beta chain, | 20138731 | 1 | HS.167531 |
| Mitochondrial precursor | | | |
| Neurogenic locus notch homolog protein 2 precursor | 20138948 | 1 | HS.487360, HS.502564 |
| Myoferlin | 20139241 | 1 | HS.500572 |
| N-acetylated-alpha-linked acidic dipeptidase II | 20139300 | 1 | HS.503560 |
| Sciellin | 20139986 | 1 | HS.115166 |
| WD-repeat protein 10 | 20140806 | 1 | HS.477537 |
| Apoptotic protease activating factor 1 | 20141188 | 1 | HS.546236 |
| Monocyte differentiation antigen CD14 precursor | 20141203 | 1 | HS.163867 |
| Elongation factor 1-delta | 20141357 | 1 | HS.333388 |
| Short chain 3-hydroxyacyl-CoA dehydrogenase, | 20141424 | 1 | HS.438289 |
| Mitochondrial precursor | | | |
| Lumican precursor | 20141464 | 1 | HS.406475 |
| Protein-glutamine gamma-glutamyltransferase | 20141877 | 1 | HS.517033 |
| Zinc finger protein 41 | 20141930 | 1 | HS.496074 |
| Complement C1q subcomponent, C chain precursor | 20178281 | 1 | HS.467753 |
| S100 calcium-binding protein A2 | 20178319 | 1 | HS.516484 |
| Lysyl-tRNA synthetase | 20178333 | 1 | HS.3100 |
| Aldo-keto reductase family 1 member C3 | 20532372 | 1 | HS.78183 |
| ATP-dependent RNA helicase DDX18 | 20532388 | 1 | HS.363492 |
| Laminin alpha-5 chain precursor | 20532393 | 1 | HS.473256 |
| Proteasome subunit beta type 3 | 20532411 | 1 | HS.82793 |
| Wnt-3a protein precursor | 20532424 | 1 | HS.336930 |
| 6-phosphogluconate dehydrogenase, decarboxylating | 20981679 | 1 | HS.464071 |
| ELAV-like protein 1 | 20981691 | 1 | HS.184492 |
| Glutathione S-transferase Mu 3 | 21264423 | 1 | HS.2006 |
| Hsp90 co-chaperone Cdc37 | 21542000 | 1 | HS.160958 |
| Ankyrin 3 | 21759000 | 1 | HS.499725 |
| Coactosin-like protein | 21759076 | 1 | HS.289092 |
| GPI transamidase component PIG-S | 21759353 | 1 | HS.462550 |
| Cadherin EGF LAG seven-pass G-type receptor 3 | 22095552 | 1 | HS.533070 |
| precursor | | | |
| ATP synthase g chain, mitochondrial | 22096328 | 1 | HS.486360 |
| Vacuolar ATP synthase catalytic subunit A, ubiquitous | 22096378 | 1 | HS.477155 |
| isoform | | | |
| SAM domain and HD domain-containing protein 1 | 22257047 | 1 | HS.472630 |
| Translocon-associated protein, alpha subunit precursor | 22261821 | 1 | HS.114033 |
| Chromodomain-helicase-DNA-binding protein 6 | 23396493 | 1 | HS.522898, HS.371979 |
| Ciliary dynein heavy chain 11 | 23396581 | 1 | HS.520245, HS.432390 |
| Kinesin-like protein KIF13B | 23396625 | 1 | HS.444767 |
| Keratin, type I cytoskeletal 16 | 23503075 | 1 | HS.432448 |
| NADP-dependent leukotriene B4 12- | 23503081 | 1 | HS.546348 |
| hydroxydehydrogenase | | | |
| Protein KIAA1404 | 23821814 | 1 | HS.371794 |
| Mucin 5B precursor | 23821885 | 1 | HS.534332, HS.534862 |
| | | | HS.523395, HS.534332 |
| Biliverdin reductase A precursor | 23830892 | 1 | HS.488143 |
| WD-repeat protein 9 | 23831562 | 1 | HS.314338 |
| Loss of heterozygosity 11 chromosomal region 2 gene A | 24211888 | 1 | HS.152944 |
| protein | | | |
| Midasin | 24212017 | 1 | HS.529948 |
| Basement membrane-specific heparan sulfate | 24212664 | 1 | HS.467545 |
| proteoglycan core protein precursor | | | |
| Exocyst complex component Sec15B | 24418685 | 1 | HS.303454 |
| PR-domain protein 11 | 25008957 | 1 | HS.147331 |
| Netrin-1 precursor | 25090820 | 1 | HS.128002 |
| Phosphatidylinositol-binding clathrin assembly protein | 25090897 | 1 | HS.163893 |
| XPA-binding protein 2 | 25091548 | 1 | HS.9822 |
| Putative GTP-binding protein PTD004 | 25453240 | 1 | HS.157351 |
| | | | HS.447547, HS.454 |
| Vacuolar protein sorting 35 | 25453321 | 1 | HS.467824 |
| Flotillin-1 | 26006960 | 1 | HS.179986 |
| Talin-2 | 26400725 | 1 | HS.511686 |
| Sulfide:quinone oxidoreductase, mitochondrial precursor | 27151704 | 1 | HS.511251 |
| Ras-related protein Rab-6C | 27734458 | 1 | HS.535586, HS.440 |
| Junctophilin 1 | 27805492 | 1 | HS.160574 |
| Bullous pemphigoid antigen 1, isoforms 6/9/10 | 27923958 | 1 | HS.485616 |
| Bullous pemphigoid antigen 1 isoforms 1/2/3/4/5/8 | 27923959 | 1 | HS.485616 |
| Zonadhesin precursor | 27924006 | 1 | HS.307004 |
| HECT domain containing protein 1 | 28380056 | 1 | HS.210850 |
| Wnt inhibitory factor 1 precursor | 29337245 | 1 | HS.284122 |
| DNA-directed RNA polymerases III 80 kDa polypeptide | 29428028 | 1 | HS.460298 |
| Nesprin 1 | 29839561 | 1 | HS.12967 |
| Nesprin-2 | 29839588 | 1 | HS.525392 |
| Map kinase phosphatase-like protein MK-STYX | 29840801 | 1 | HS.11615 |
| Putative Polycomb group protein ASXL1 | 30172872 | 1 | HS.374043 |
| GDNF family receptor alpha 4 precursor | 30173123 | 1 | HS.302025 |
| Glucosamine--fructose-6-phosphate aminotransferase | 30923274 | 1 | HS.468864 |
| [isomerizing] 1 | | | |
| Programmed cell death 6-interacting protein | 31076831 | 1 | HS.475896 |
| Egl nine homolog 3 | 32129515 | 1 | HS.135507 |
| ADAMTS-9 precursor | 32130427 | 1 | HS.476604 |
| Myosin Id | 32172416 | 1 | HS.462777 |
| GRIP and coiled-coil domain-containing protein 2 | 32469733 | 1 | HS.436505 |
| Dedicator of cytokinesis protein 3 | 32469734 | 1 | HS.476284 |
| Transcription elongation factor B polypeptide 1 | 32699511 | 1 | HS.546305 |
| Early endosome antigen 1 | 34222508 | 1 | HS.506309 |
| X-linked interleukin-1 receptor accessory protein-like 1 | 34222654 | 1 | HS.495893 |
| precursor | | | |
| HLA class I histocompatibility antigen, A-3 alpha chain | 34223717 | 1 | HS.181244 |
| precursor | | | |
| Zinc finger protein 430 | 34925658 | 1 | HS.466289 |
| Heat shock 70 kDa protein 6 | 34978357 | 1 | HS.3268 |
| Sphingosine-1-phosphate lyase 1 | 37999486 | 1 | HS.499984 |
| Proteasome subunit alpha type 5 | 38258905 | 1 | HS.485246 |
| Zinc finger protein 397 | 38258943 | 1 | HS.464896 |
| Unc-112 related protein 2 | 41018464 | 1 | HS.180535 |
| Piccolo protein | 41019528 | 1 | HS.12376, HS.455230 |
| Cohen syndrome protein 1 | 42558898 | 1 | HS.191540 |
| Liprin-alpha 1 | 42558969 | 1 | HS.530749 |
| Hornerin | 45476906 | 1 | |
| Zinc finger protein ZFPM2 | 45476962 | 1 | HS.431009 |
| Eukaryotic initiation factor 4A-II | 45645183 | 1 | HS.478553 |
| Protein MICAL-2 | 46396148 | 1 | HS.501928 |
| Low-density lipoprotein receptor-related protein 10 | 46396347 | 1 | HS.525232 |
| precursor | | | |
| Periphilin 1 | 46396942 | 1 | HS.444157 |
| Vesicle trafficking protein SEC22b | 46397702 | 1 | HS.534212 |
| Ras-related protein Rab-10 | 46577638 | 1 | |
| Ubiquitin-conjugating enzyme E2 D3 | 46577654 | 1 | HS.518773, HS.472031 |
| Ras-related protein Rab-25 | 46577696 | 1 | HS.491308 |
| Galectin-3 binding protein precursor | 47115668 | 1 | HS.514535 |
| Tubulin tyrosine ligase-like protein 2 | 47117620 | 1 | |
| Actin-like protein 2 | 47117648 | 1 | HS.393201 |
| 40S ribosomal protein S3a | 47117764 | 1 | HS.356572 |
| Nuclear receptor corepressor 1 | 47117817 | 1 | HS.462323, HS.307 |
| Dynamin-2 | 47117856 | 1 | HS.211463 |
| Histone H2A.q | 47117890 | 1 | |
| Transforming protein RhoA | 47606458 | 1 | HS.247077 |
| Scavenger receptor class F member 2 precursor | 47606791 | 1 | HS.474251 |
| Defender against cell death 1 | 48428858 | 1 | HS.82890 |
| Signal transducer and activator of transcription 3 | 48429227 | 1 | HS.463059 |
| Pantophysin | 48474786 | 1 | HS.80919 |
| Serine/threonine protein phosphatase PP1-beta | 49065814 | 1 | HS.468018 |
| catalytic subunit | | | |
| 40S ribosomal protein S7 | 49065831 | 1 | HS.546287, HS.534 |
| | | | HS.33348 |
| Inositol hexaphosphate kinase 1 | 50400597 | 1 | HS.438691 |
| Ninein | 50400772 | 1 | HS.310429 |
| Potassium channel tetramerisation domain containing | 50401124 | 1 | HS.109438 |
| protein 12 | | | |
| Signal-induced proliferation-associated 1 like protein 1 | 50401319 | 1 | HS.208846 |
| Signal-induced proliferation-associated 1 like protein 2 | 50401690 | 1 | HS.268774 |
| Ras-related protein Rab-11A | 50402542 | 1 | HS.321541 |
| 40S ribosomal protein S18 | 50403625 | 1 | HS.546290 |
| Receptor-type tyrosine-protein phosphatase S precursor | 50403770 | 1 | HS.408456 |
| Bassoon protein | 51315800 | 1 | HS.194684 |
| Drebrin-like protein | 51316115 | 1 | |
| Unc-13 homolog D | 51316668 | 1 | HS.41045 |
| ADP-ribosylation factor 6 | 51316984 | 1 | HS.525330 |
| ADP-ribosylation factor 1, ADP-ribosylation factor | 51316985 | 1 | HS.286221 |
| 3, ADP-ribosylation factor 5 | | | |
| Guanine nucleotide-binding protein G(I)/G(S)/G(T) beta | 51317302 | 1 | HS.430425 |
| subunit 1 | | | |
| Ras-related protein Rap-1A | 51338607 | 1 | HS.190334 |
| 60S ribosomal protein L23 | 51338639 | 1 | HS.406300, HS.512542 |
| Small nuclear ribonucleoprotein Sm D2 | 51338666 | 1 | HS.515472 |
| F-box only protein 44 | 51338823 | 1 | HS.519716 |
| Histone H2B K | 51701495 | 1 | HS.437275 |
| Zinc finger protein 237 | 51702202 | 1 | HS.530988 |
| Zinc finger protein 330 | 51702204 | 1 | HS.120766 |
| 60S ribosomal protein L11 | 51702795 | 1 | HS.388664 |
| Vesicle-associated membrane protein 2 | 51704192 | 1 | HS.25348 |
| Puromycin-sensitive aminopeptidase | 51704228 | 1 | HS.443837 |
| Interferon-induced 17 kDa protein precursor | 52001470 | 1 | HS.458485 |
| Protein FAM49B | 52782794 | 1 | HS.492869 |
| Kelch-like protein 17 | 52783052 | 1 | HS.109212 |
| 60S ribosomal protein L38 | 52783779 | 1 | HS.380953 |
| Actin, cytoplasmic 2 | 54036678 | 1 | HS.514581 |
| Actin, alpha cardiac | 54036697 | 1 | HS.118127 |
| Neutral alpha-glucosidase AB precursor | 54037162 | 1 | HS.76847 |
| Eukaryotic translation initiation factor 5A | 54037409 | 1 | HS.534314 |
| Ubiquitin-conjugating enzyme E2 L3 | 54039805 | 1 | HS.108104 |
| Annexin A7 | 55584155 | 1 | HS.386434 |
| Kin of IRRE-like protein 3 precursor | 55736065 | 1 | HS.302350 |
| Tripartite motif protein 29 | 55976299 | 1 | HS.504115 |
| Vesicle-associated membrane protein 8 | 55976764 | 1 | HS.534373 |
| Abnormal spindle-like microcephaly-associated protein | 55976785 | 1 | HS.121028 |
| Tubulin beta-2 chain | 55977480 | 1 | HS.433615 |
| Tubulin alpha-3 chain | 55977864 | 1 | HS.524395 |
| Zinc finger protein 219 | 55977885 | 1 | HS.250493 |
| Netrin-2 like protein precursor | 56404431 | 1 | HS.158336 |
| Zinc finger protein 644 | 56404958 | 1 | HS.173001 |
| Serine/threonine-protein kinase 38-like | 56749668 | 1 | |
| Hemoglobin gamma-2 chain | 56749861 | 1 | |
| Keratin, type II cytoskeletal 5 | 56757580 | 1 | |
| Dynein heavy chain, cytosolic | 57015308 | 1 | |
| Low-density lipoprotein receptor-related protein 1B | 57015418 | 1 | |
| precursor | | | |
| DNA polymerase eta | 59798441 | 1 | |
| TBC1 domain family member 21 | 59798963 | 1 | |
| Keratin, type II cytoskeletal 6E | 59803089 | 1 | |
| Protein C19orf10 precursor | 61221730 | 1 | |
| Autophagy protein 7-like | 62286592 | 1 | |
| Delta-1-pyrroline-5-carboxylate dehydrogenase, | 62511241 | 1 | |
| Mitochondrial precursor | | | |
| Interleukin enhancer-binding factor 3 | 62512150 | 1 | |
| Enoyl-CoA hydratase, mitochondrial precursor | 62906863 | 1 | |
| ATP synthase g chain, mitochondrial | 62906882 | 1 | |
| Keratin, type II cytoskeletal 1b | 66774007 | 1 | |
| Thymidine phosphorylase precursor | 67477361 | 1 | |
| Exportin-1 | 68052989 | 1 | |
| Cathepsin B precursor | 68067549 | 1 | |
| Myosin-14 | 71151982 | 1 | |
| ATP-dependent RNA helicase A | 71153504 | 1 | |

TABLE 4
Relative Abundant Proteins Downregulated in High-Grade Cervical Specimens

| | | Number Down- |
|---|---|---|
| Gene/Protein Name | GI Accession | Regulated Pairs |
| Calmodulin-related protein NB-1 | 115502 | 7 |
| Interleukin-1 receptor antagonist protein precursor | 124312 | 7 |
| Arachidonate 12-lipoxygenase, 12S-type | 126400 | 7 |
| Desmoplakin | 12644130 | 7 |
| Periplakin | 14195005 | 7 |
| Small proline-rich protein 3 | 20138065 | 7 |
| Plakophilin 3 | 20139301 | 7 |
| Keratin, type II cytoskeletal 4 | 20141510 | 7 |
| Squamous cell carcinoma antigen 1 | 20141712 | 7 |
| Desmoglein-3 precursor | 416918 | 7 |
| Involucrin | 124731 | 6 |
| Keratin, type II cytoskeletal 3 | 125098 | 6 |
| Hurpin | 12643252 | 6 |
| Cystatin B | 1706278 | 6 |
| Plakophilin 1 | 20138951 | 6 |
| Aldo-keto reductase family 1 member B10 | 20531983 | 6 |
| Mucin 5B precursor | 23821885 | 6 |
| Chloride intracellular channel protein 3 | 46397812 | 6 |
| Puromycin-sensitive aminopeptidase | 51704228 | 6 |
| Maspin precursor | 547892 | 6 |
| Junction plakoglobin | 130257 | 5 |
| Retinoic acid-binding protein II, cellular | 132401 | 5 |
| Keratin, type II cytoskeletal 6A | 1346344 | 5 |
| Galectin-7 | 1346431 | 5 |
| Envoplakin | 14194715 | 5 |
| Fatty acid-binding protein, epidermal | 232081 | 5 |
| Desmoglein-1 precursor | 416917 | 5 |
| Desmocollin 2A/2B precursor | 461968 | 5 |
| Protein-glutamine gamma-glutamyltransferase K | 57015359 | 5 |
| Keratin, type I cytoskeletal 13 | 6016411 | 5 |
| Ig alpha-1 chain C region | 113584 | 4 |
| Cystatin A | 118177 | 4 |
| Placental ribonuclease inhibitor | 132573 | 4 |
| Keratin, type II cytoskeletal 6B | 1346345 | 4 |
| Protein-glutamine glutamyltransferase E precursor | 13638501 | 4 |
| Low-density lipoprotein receptor-related protein 1 precursor | 1708865 | 4 |
| Squamous cell carcinoma antigen 2 | 1710877 | 4 |
| Airway trypsin-like protease precursor | 17376886 | 4 |
| Sciellin | 20139986 | 4 |
| S100 calcium-binding protein A14 | 20178118 | 4 |
| Tubulin alpha-6 chain | 20455322 | 4 |
| Antileukoproteinase 1 precursor | 113636 | 3 |
| Annexin A1 | 113944 | 3 |
| Annexin A3 | 113954 | 3 |
| Annexin A8 | 113967 | 3 |
| Carbonyl reductase | 118519 | 3 |
| Ezrin | 119717 | 3 |
| Gelsolin precursor | 121116 | 3 |
| Histone H2A.g | 121978, 121959, | 3 |
| | 12585257 | |
| Keratin, type I cytoskeletal 15 | 125081 | 3 |
| Ig kappa chain V-III region SIE, Ig kappa chain V-III region WOL | 125797, 125803 | 3 |
| Phosphoglycerate mutase 1 | 130348 | 3 |
| Fatty acid synthase | 1345959 | 3 |
| Breast cancer type 2 susceptibility protein | 14424438 | 3 |
| Ketohexokinase | 1730044 | 3 |
| Polymeric-immunoglobulin receptor precursor | 1730570 | 3 |
| Serpin B12 | 20140145 | 3 |
| 6-phosphogluconate dehydrogenase, decarboxylating | 20981679 | 3 |
| Leukocyte elastase inhibitor | 266344 | 3 |
| Long palate, lung and nasal epithelium carcinoma associated | 34395685 | 3 |
| protein 1 precursor | | |
| Eukaryotic initiation factor 4A-I | 46397463 | 3 |
| Ras-related protein Rab-2A | 46577636 | 3 |
| Keratin, type II cuticular HB2 | 48474984 | 3 |
| ERO1-like protein alpha precursor | 50400608 | 3 |
| ADP-ribosylation factor 1, ADP-ribosylation factor 3 | 51316985, 47117657 | 3 |
| Tubulin alpha-ubiquitous chain | 55977474 | 3 |
| Tubulin beta-2 chain | 55977480 | 3 |

Criteria for inclusion in upregulated sample pairs HSIL/WNL (Total N = 11):
1) Cutoff values of >4 fold or >4s in total peptide ratios
2) >2.5 fold for software integration analysis (specimen pairs 48/65, 51/66, 50/80)
3) Quantitative data >1.7
Criteria for inclusion in downregulated sample pairs WNL/HSIL (Total N = 11):
1) Cutoff values of >4 fold or >4s in total peptide ratios
Table 1: Selected proteins with significant biological interest upregulated in 3+ specimen pairs
Table 2: Relative abundant proteins upregulated in 3+ specimen pairs
Table 3: Lower probability proteins upregulated in 1 or 2 specimen pairs
Table 4: Relative abundant proteins downregulated in 3+ specimen pairs

### REFERENCES

ASCUS-LSIL Triage Study (ALTS) Group (2003), Am J Obstet Gynecol, 188:1383-92 and 1393-400.
Baldwin et al. (2003) Nature Reviews Cancer 3:1-10.
Crook et al. (1991) Cell 67(3):547-56.
Cuschieri et-al. (2005) Journal of Clinical Pathology 58:946-950.
Howell et al. (2004) Diagn Cytopathol, 30(5):362-6.
Joste et al. (2005) Diagnostic Cytopathology 32(5):310-314.
Melnikow et al. (1998) Natural history of cervical squamous intraepithelial lesions: a meta-analysis. Obstet Gynecol 92:727-35.
Munoz et al. (1989) Epidemiology of Cervical Cancer In: “Human Papillomavirus,” New York, Oxford Press, pp 9-39.
National Institutes of Health, Consensus Development Conference Statement on Cervical Cancer, Apr. 1-3, 1996.
Roberts et al. (1997) The Medical Journal of Australia 167:466-469.
Shai et al. (2007) Cancer Research 67:1626-1635.
Shiffman et al. (2003) Arch Pathol Lab Med 127(8):946-9.
Syrjanen et al. (1987) Applied Pathol 5:121-135.
Tabbara et al. (1992) The Bethesda classification for squamous intraepithelial lesions: histologic, cytologic and viral correlates, Obstet Gynecol 79:338-346.

While the present invention has been described in conjunction with a preferred embodiment, one of ordinary skill, after reading the foregoing specification, will be able to effect various changes, substitutions of equivalents, and other alterations to the compositions and methods set forth herein. It is therefore intended that the protection granted by Letters Patent hereon be limited only by the definitions contained in the appended claims and equivalents thereof.

1. A method for assessing the presence of a cervical dysplastic lesion in a human subject, said method comprising comparing:
the level of abundance, in a sample from said subject, of at least one marker selected from the group consisting of the markers listed in Tables 1-4; and
the normal level of abundance of said at least one marker in a control sample,
wherein a significantly higher level of abundance of said at least one marker in said sample from said subject compared to the level of abundance of said at least one marker in said control sample is an indication of the presence of a cervical dysplastic lesion in said subject.

2. The method of claim 1, wherein said significantly higher level of abundance is three or more times the abundance level of said at least one marker in said control sample.

3. The method of claim 1, wherein the level of abundance of said at least one marker is determined by detecting the amount of marker protein present in the sample.

4. The method of claim 1, wherein the level of abundance of said at least one marker is determined using an assay selected from the group consisting of an antibody based assay, a protein array assay and a mass spectrometry based assay.

5. The method of claim 1, wherein said control sample level of abundance of said at least one marker is determined from a standard table or curve.

6. The method of claim 1, wherein the level of abundance of said at least one marker is determined by detecting the amount of mRNA that encodes a marker protein present in the sample.

7. The method of claim 1, wherein said at least one marker is a plurality of markers.

8. The method of claim 7, wherein said plurality of markers is greater than three.

9. The method of claim 7, wherein said plurality of markers is greater than five.

10. A method of selecting a composition for inhibiting cervical dysplasia in a patient, the method comprising the steps of:
a) obtaining a sample comprising cervical dysplastic cells from a patient;
b) separately exposing a plurality of specimens from said sample to a plurality of test compositions;
c) following said exposing steps, comparing the relative level of abundance of a plurality of markers in each specimen of said sample, wherein at least two of the markers are selected from the group consisting of markers listed in Tables 1-4; and
d) selecting at least one of the test compositions that modifies the relative level of abundance of the plurality of markers in the aliquot exposed to that test composition, compared to the other test compositions, as said composition for inhibiting cervical dysplasia in said patient.

11. A kit for assessing the presence of a cervical dysplastic lesion in a human subject, the kit comprising reagents for carrying out the method of claim 1.

12. A kit for assessing the presence of a cervical dysplastic lesion in a human subject, the kit comprising a plurality of antibodies, wherein at least two of the antibodies specifically bind with proteins corresponding to at least two markers selected from the group consisting of markers listed in Tables 1-4.

13. A kit for assessing the suitability of one or more test compounds for inhibiting cervical dysplasia in a patient, the kit comprising: a) one or more test compounds; and b) a reagent for assessing the relative level of abundance of a plurality of markers, wherein at least two of the markers are selected from the group consisting of markers listed in Tables 1-4.

14. A method for assessing the presence of a cervical dysplastic lesion in a human subject, said method comprising the steps of:
a) identifying a human subject to be screened for a cervical dysplastic lesion;
b) providing a cervical sample from said subject;
c) determining the level of abundance in said subject sample of at least one marker selected from the group consisting of the markers listed in Tables 1-4;
d) determining the level of abundance of said at least one marker in a control sample; and
e) comparing the level of abundance of said at least one marker in the subject sample to the level of abundance of said at least one marker in the control sample,
wherein a significantly higher level of abundance of said at least one marker in said subject sample compared to the level of abundance of said at least one marker in said control sample is an indication of the presence of a cervical dysplastic lesion in said subject.
